(19) United States
(12) Patent Application Publication
(10) Pub. No.: US 2005/0221482 A1
(43) Pub. Date: Oct. 6, 2005

(54) Methods and compositions for obtaining hematopoietic stem cells derived from embryonic stem cells and uses thereof

(76) Inventors: Richard K. Burt, Evanston, IL (US); Larissa Verda, Evanston, IL (US); Charles J. Link JR., Ames, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 11/094,977

(22) Filed: Mar. 31, 2005

Related U.S. Application Data
(60) Provisional application No. 60/558,018, filed on Mar. 31, 2004.

(51) Int. Cl. C12N 5/08; C12N 5/06

(52) U.S. Cl. 435/372; 435/354

(57) ABSTRACT

The present invention provides an isolated population of adult hematopoietic stem cells (HSC) derived from embryonic stem cells (ESC) induced to differentiate in vitro by culturing ESCs in a medium with stem cell factor, interleukin (IL)-3, and IL-6. HSC of immunophenotype c-kit⁺ or c-kit+/CD45⁺ from this population are isolated and injected either intra bone marrow or intravenously into myeloablated recipient individuals. This method allows for the establishment of banks of allogeneic ESC-derived adult stem cells for treatments of autoimmune diseases, immune deficiencies and induction of immunotolerance during organ transplantation. These allogeneic ESC-derived adult hematopoietic stem cells (HSC) may be used in reconstituting bone marrow, without the development of teratomas or graft versus host disease, despite crossing histocompatibility barriers. Additionally, allogeneic ESC-derived HSC can be used to prevent the development of autoimmune diseases or organ rejection during transplantation.

### CROSS-REFERENCE TO RELATED APPLICATIONS

This application claims the priority benefit of U.S. provisional patent application 60/558,018, filed Mar. 31, 2004. The priority application is incorporated herein by reference in its entirety.

### FIELD OF THE INVENTION

The present method belongs to the field of bone marrow transplantation, embryonic stem cell differentiation, reconstitution of a functional immune system and induction of immunotolerance. The method can be applied to reconstitute multilineage hematopoiesis and a functional immune system without the induction of teratomas or graft versus host disease for the treatment of conditions that are associated or require partial or total myeloablation followed by bone marrow transplantation, such as leukemias, autoimmune diseases, immunodeficiencies, or cancer chemotherapy.

### BACKGROUND OF THE INVENTION

Hematopoietic stem cells (HSCs) obtained from the marrow or peripheral blood are being used worldwide to treat malignancies, inborn errors of metabolism, and autoimmune diseases (1-3). However, this relies on a supply of genetically compatible bone marrow donors. Attempts to maintain HSCs in culture for even relatively short periods of time are unsuccessful due to terminal differentiation, which precludes the establishment of a collection of HSC lines or cell banks of different histocompatibility types that can be used as a universal source of donor HSCs. In addition, a common morbid and/or lethal complication of HSC transplantation using bone marrow from allogeneic donors with mismatched histocompatibility is the development of graft versus host disease (GVHD) (4, 5), and/or host versus graft, which results in rejection of the graft. Several approaches for suppressing the immune response of recipients towards the grafts have been attempted by treatment with immunosuppressive drugs or radiation, but these treatments are costly and often have side effects. A typical bone marrow graft composition includes T cells, dendritic cells, B cells, and CD34⁺ or other progenitor cells. This composition varies depending on the patient, the donor source, the harvesting technique and can suffer from different grades of bacterial contamination. This has resulted in intra-institutional and inter-institutional variation in graft composition. For these reasons, a renewable source of HSCs that is not complicated by GVHD and does not have interpatient, intrapatient, or lot variability would be highly desirable.

Embryonic stem cell (ESC) lines are derived from the inner cell mass of the blastocyst and are totipotent and immortal. A single embryonic stem cell (ESC) line can be repetitively cryopreserved, thawed, expanded, and differentiated into various cellular components serving as a potentially renewable and well characterized source of adult stem cells. ESCs can be expanded ex vivo as undifferentiated cells that retain a normal karyotype or, alternatively, can be differentiated ex vivo into cell types of all three germ layers by changing the culture conditions or exposing the cells to different combinations of growth and differentiation factors (6, 7). Unfortunately, ESCs cannot be directly used as a source of stem cells for in vivo treatments as their uncontrolled in vivo proliferation and differentiation results in the development of teratomas. Consequently, ESCs need to be differentiated ex vivo into adult stem cells of a defined tissue type for therapeutic applications.

Mouse ESCs can be maintained in undifferentiated state by incubation with Leukemia inhibitory factor (LIF). Withdrawal of LIF initiates the formation of embryoid bodies (EB) and cellular differentiation (8, 9). When ESCs are used to produce desired cells, it is often preferable to optimize differentiation towards specific cell types. In the particular case of differentiation of ESC into adult hematopoietic stem cells it is desirable that the resulting hematopoietic stem cells can originate multiple hematopoietic lineages. When EB are cultured, cells with hematopoietic progenitor phenotype are routinely observed in vitro (10-14). In the absence of cytokines or stromal cells, multilineage hematopoietic precursors might be detected by colony-forming assays after 4 d of EB culture. C-kit (stem cell factor [SCF] receptor) and CD45 (a hematopoietic lineage marker) expression occur simultaneously on day 10 of EB culture (15).

There have been some attempts to direct murine embryonic cell populations toward differentiation into hematopoietic cells. For example, in U.S. Pat. Nos. 6,280,718 and 6,613,568 it is described a method to induce differentiation of ESCs into hematopoietic cells by culturing the ESCs on a layer of irradiated stromal cells collected from the yolk sac of mice at embryonic day 12, or onto a layer of stromal cells obtained from mouse bone marrow. However, the repopulating ability of these in vitro differentiated hematopoietic cells of multiple lineages has not been demonstrated in vivo. Moreover, stromal cells obtained from the yolk sac of embryos are required to induce the differentiation of ESCs into multilineage hematopoietic cells, which imposes a practical limitation if this method was to be applied as a source of alternate bone marrow transplantation in humans. Mouse ES cell embryoid bodies form blood islands capable of the generation of lymphoid and myeloid mixed-cell populations when cultured in vitro (38). The in vitro derivation of hematopoietic cells from mouse ESCs is enhanced by addition of stem cell factor (SCF), IL-3, IL-6, IL-11, GM-CSF, EPO, M-CSF, G-CSF, LIF, and recapitulates mouse E6.5 to 7.5 hematopoietic development (39-41). Murine ESCs can also generate hematopoietic stem cells when cultured on a stromal cell line in the presence of IL-3, IL-6 and fetal liver stromal cell line cultured supernatant. It is not clear what proportion of ESCs cultured onto stromal cells and differentiated into hematopoietic cells are true hematopoietic stem cells with multilineage regeneration potential.

The use of hematopoietic cells derived from ESCs has been envisioned by others as an alternative source of bone marrow transplantation. However, the conception of the idea generally involves the use of ESC lines that are compatible with the major histocompatibility complex (MHC) of the recipient, in order to avoid GVHD or rejection of the graft. Preserving the requirement of MHC compatibility is not always possible and it would require having a catalogued transplant depository of ESCs derived from multiple donors, each of the ESCs being homozygous for a unique HLA haplotype, for the purpose of having a constant, reliable and comprehensive supply of immunohistocompatible cells for diagnosis, treatment and/or transplantation. Alternatives to the establishment of such a collection of ESCs has been mentioned by others, such as methods to use the recipient's cell nucleus as a source of the genetic material for generation of genetically identical ESCs have been presented. For example, WO 98/07841 discusses techniques of deriving embryonic stem cells that are MHC compatible with a selected donor by transplanting a nucleus obtained from the recipient into an enucleated oocyte obtained from a donor, followed by derivation of the embryonic stem cells. The application suggested that the resulting cells could be used to obtain MHC compatible hematopoietic stem cells for use in medical treatments requiring bone marrow transplantation. However, this method requires the somatic cloning of the donor genetic material by nuclear transfer into donor oocytes, followed by generation of embryos from which embryonic stem cells are derived which are subsequently induced to differentiate into several lineages such as hematopoietic cells. This method has several technical and ethical limitations when applied to human beings and clearly, methods that do not rely on human cloning would be desirable.

In this sense, alternative methods to bone marrow transplantation that would allow the regeneration of a fully functional immune system after partial or total myeloablation procedure, without the need of MHC compatibility between the donor and recipient, without the risk of triggering graft vs host disease (GVHD) frequently associated with MHC incompatibility, and using standardized cell preparations and procedures, would be highly desirable. An additional challenge for developing cell therapies from ESCs is whether in vitro differentiated hematopoietic stem cells can adapt to function effectively in vivo when transplanted into an adult and reconstitute a functional immune system (16).

Therefore, there is a need for the present invention.

Thus, it is a primary object, feature, or advantage of the present invention to improve upon the state of the art.

It is a further object, feature, or advantage of the present invention to provide a renewable source of healthy tissue stem cells for all organ systems.

It is a further object, feature, or advantage of the present invention to provide an isolated population of adult hematopoietic stem cells (HSC) for the treatment of autoimmune diseases and immunodeficiencies.

It is a further object, feature, or advantage of the present invention to provide a method of reconstituting an immune system that promotes immunotolerance of an allogeneic donor.

It is a further object, feature, or advantage of the present invention to provide a method of reconstituting bone marrow without the development of teratomas.

It is a further object, feature, or advantage of the present invention to provide a method of reconstituting bone marrow without the development of graft versus host disease.

It is a further object, feature, or advantage of the present invention to provide a method of preventing the rejection of an allogeneic organ during transplantation.

These and other objects, features, or advantages will become apparent from the following description of the invention.

### SUMMARY OF THE INVENTION

The present invention provides an isolated population of adult hematopoietic stem cells that display a c-kit CD117 cell surface marker that proliferates in culture and methods of use therefor.

Accordingly, among its various aspects, the present invention provides an isolated population of cells produced by the following method: culturing an embryonic stem cell in a medium that comprises at least one growth factor so that said cell forms a population of cells; and selecting from said population, cells displaying a c-kit CD117 cell surface specific marker, thereby isolating a population of cells that are c-kit CD117 positive.

In another embodiment, the present invention provides a method of obtaining adult hematopoietic stem cells, comprising: culturing an embryonic stem cell in a medium comprising a hematopoietic growth factor; so that said cell forms a population of cells; and selecting from said population cells displaying a c-kit CD117 cell surface specific marker.

In yet another embodiment, the present invention provides a method of obtaining adult hematopoietic stem cells comprising: culturing an embryonic stem cell in a medium with a growth factor selected from a group consisting of: at least one of the following: stem cell factor (SCF), interleukin-3 (IL-3), and interleukin-6 (IL-6), so that said cell forms a population of cells; and selecting from said population cells displaying a c-kit CD117 cell surface specific marker, thereby isolating a population of cells that are c-kit CD117 positive.

In yet another embodiment, the present invention provides a method of reconstituting or supplementing hematopoietic cell function in a recipient subject comprising: obtaining adult hematopoietic stem cells comprising: culturing an embryonic stem cell in a medium comprising at least one of the following: stem cell factor (SCF), interleukin-3 (IL-3), or interleukin-6 (IL-6), so that said cell forms a population of cells; and selecting from said population of cells that are c-kit CD117 positive; administering said selected c-kit CD117 positive cells into a recipient subject.

In yet another embodiment, the present invention provides a method of promoting immunotolerance in a recipient subject to a cell population that is allogeneic to a recipient subject's comprising: obtaining adult hematopoietic stem cells (HSC) produced by the method comprising: culturing an embryonic stem cell in a medium comprising at least one of the following: stem cell factor, interleukin-3 or interleukin-6, so that said cell forms a population of cells; and selecting from said population cells that are c-kit CD117 positive, and administering the selected c-kit CD117 positive cells into a recipient subject; thereby promoting immunotolerance to cells syngeneic to the transplanted HSC.

In yet another embodiment, the present invention provides a method of preventing or decreasing cell mediated graft versus host disease (GVHD) and/or host versus graft disease (HVGD) derived from an MHC incompatible donor in a recipient of the transplant, the method comprising: obtaining adult hematopoietic stem cells produced by the method comprising: culturing an embryonic stem cell in a medium comprising at least one of the following: stem cell factor, interleukin-3 or interleukin-6, so that said cell forms a population of cells; and selecting from said population of cells, those cells that are c-kit CD117 positive, and administering the selected c-kit CD117 positive cells into a recipient subject; thereby promoting immunotolerance to said cells, thereby preventing or decreasing cell mediated GVHD and graft rejection of the transplant.

In yet another embodiment, the present invention provides a method of treating autoimmune type I diabetes comprising: obtaining adult hematopoietic stem cells produced by the method comprising: culturing an embryonic stem cell in a medium comprising at least one of the following: stem cell factor, interleukin-3 or interleukin-6, so that said cell forms a population of cells; and selecting from said population cells that are c-kit CD117 positive; and transplanting into a bone marrow cavity of a myeloablated recipient subject with autoimmune type I diabetes a therapeutic amount of selected c-kit CD117 positive cells adult hematopoietic stem cells.

### BRIEF DESCRIPTION OF THE DRAWINGS

FIG. 1. Immunophenotype of undifferentiated ESCs. Expression of c-kit, CD45 (b), and CD34 and H2^{b} (d) compared with isotype control (a) and normal murine bone marrow (c).

FIG. 2. Immunophenotype of cytokine-stimulated ESCs. Percent of cytokine-stimulated ESCs that are c-kit⁺ (a) and CD45⁺ (b) cells. Immunophenotypic characteristics of ESC-derived cells sorted for dual c-kit⁺ CD45⁺: Sca-1⁺ and c-kit⁺ (c), H2^{b+} and c-kit⁺ (d), CD45⁺ and c-kit⁺ (e), and Lin⁻ (f-h).

FIG. 3. Cytokine-stimulated ESCs ex vivo and in vitro analysis data. (a) Efficiency of hematopoietic colony formation by 200 ESC-derived cells (different population: enriched for ckit⁺, c-kit⁺ CD45⁺, and CD34⁺). (b) Survival curve for mice injected i.v. with non-sorted ESC-derived cells, IBM injected with non-sorted ESC-derived cells, and injected i.v. or IBM with sorted c-kit⁺ CD45⁺ cells. (c) Mean percentage of donor chimerism in different groups of mice analyzed 2, 4, 10, and 20 wk after ESCT. IBM-5.5, irradiated with 5.5 Gy, injected IBM; IBM-8, irradiated with 8.0 Gy, injected IBM; i.v.-8, irradiated with 8.0 Gy, injected i.v.

FIG. 4. Immunophenotype of peripheral blood after ESC transplantation. Comparison of percentage of H2^{b+} leukocytes in peripheral blood of the C57BL/6J mouse (a), BALB/c mouse (b), and chimeric BALB/c mouse 2 wk after ESC-transplantation (TBI 8.0 Gy/TBI; c). Example of analysis of chimerism based on immunophenotyping of PBMCs in two channels: H2^{b} (donor-derived) and H2^{d} (host-derived) and CD45⁺ (10 wk after ESCT; TBI 8.0 Gy/TBI; d and e). Analysis of H2^{b+} mononuclear cells in peripheral blood from chimeric mouse (20 wk after ESCT; TBI 8.0 Gy/IBM): gating (H gate) positive population H2^{b+} (f) and analysis of percent of T lymphocytes (CD3⁺), B lymphocytes (CD19⁺; g), and granulocytes/monocytes (CD11b⁺/CD14⁺; h).

FIG. 5. Immunologic competence of ESC-derived hematopoiesis. (a) Proliferative response of splenocytes from chimeric mice to donor and recipient MHC and third party antigen (data are presented as a percent of BrdU incorporated cells). (b) Production of IFN-γ during mixed lymphocyte reaction (MLR) analyzed by ELISA (mean values). (c) Correlations between donor chimerism analyzed at 20 wk after transplantation and proliferative response to donor MHC (analyzed by MLR) and (d) IFN-γ level assessed by ELISA in MLR (donor and chimeric splenocytes) supernatant. Proliferative response and IFN-γ data are presented in log scale, whereas chimerism is shown as the percentage of donor (ESC-derived) cells in peripheral blood in linear scale.

FIG. 6. NOD mice survival curve. Three groups of mice were followed up for 38 weeks. Once group received intra-bone marrow (IBM) injection of ESC-derived HSC (n=10), another group received intravenous (IV) injection of ESC-derived HSC (n=8) and 9 mice were held untreated as controls.

FIG. 7. In vitro response of splenocytes to GAD65 (analyzed by INFg level in supernatant after 72 h of culture, by ELISA). Y-axis represents the IFNg level in pg/ml. X-axis represents different treatment groups: 1) ESCT-ST, splenocytes from non-obese diabetic (NOD) mice transplanted with ESCs-derived HSC, stimulated; 2) ESCT-N, splenocytes from NOD mice transplanted with ESCs-derived HSC, not stimulated; 3) NOD-ST, splenocytes from NOD mice, stimulated; 4) NOD-N, splenocytes from NOD mice not stimulated; 5) B6-ST, splenocytes from C57BL/6 mice, stimulated (negative control); and 6) B6-N, splenocytes from C57BL/6 mice, not stimulated (negative control).

FIG. 8. Mixed lymphocyte culture response data analyzed by BrdU incorporation. Splenocytes were cultured for 96 h in the following combinations: 1) B_B: C57BL/6 with irradiated C57BL/6 (negative control); 2) NOD_B: NOD with irradiated C57BL/6; 3) E_{—}129: NOD transplanted with ESC-derived HSC with irradiated 129Sv (ESC origin); 4) E_B: NOD transplanted with ESC-derived HSC with irradiated C57BL/6 (third party).

FIG. 9. Histological analyses of pancreases (hematoxilin & eosin staining, 40×, panels A, C, E, G and I) and immunohistochemical analyses of islet cells for insulin (staining for insulin, 40×, panels B, D, F, H and J). Panels A and B show staining from NOD mice with symptoms of diabetes (positive control). Panels C and D show staining from C57BL/6 mouse (normal control). Panels E to J show staining of pancreases from NOD mice transplanted with ESC-derived HSCs.

FIG. 10. Immunophenotype of expanded in vitro mesenchymal cells derived from bone marrow ESC transplantation in mice. A—isotype control, B, C, D—analyses of ESC transplantations—1, 2 and 3 chimeric mice derived bone marrow stromal cells.

### DETAILED DESCRIPTION OF THE PREFERRED EMBODIMENT

The present invention involves the production of a population of adult hematopoietic stem cells that are differentiated from at least one embryonic stem cell and from which cells are selected for those cells displaying c-kit CD117 cell surface marker. This population can then be used in reconstituting or supplementing hematopoietic cell function in a recipient subject, promoting immunotolerance in a recipient subject, preventing or decreasing the occurrence of cell mediated graft versus host disease (GVHD) and teratomas in a recipient subject, and treating autoimmune type I diabetes in a recipient subject.

Definitions

As used herein, the term “a population” refers to one or more cells.

As used herein, “embryonic stem cell” refers to a cell that can give rise to many differentiated cell types in an embryo or an adult, including the germ cells (sperm and eggs). Embryonic stem cells are also capable of self-renewal, and are derived from the inner mass of the blastocyst. This cell type is also referred to as an “ES cell” or “ESC” herein. This invention makes use of pluripotential ES cell which can be maintained in undifferentiated state while growing on feeder layers and give rise to embryoid bodies and multiple differentiated cell phenotypes in monolayer culture after change of the culture conditions. Given the methods described herein, an ES cell can be made for any animal. However, mammals are preferred since many beneficial uses of mammalian ES cells exist. Mammalian ES cells such as those from mouse, rat, rabbit, guinea pig, goat, pig, cow, and human can be obtained.

As used herein, “hematopoietic stem cell” refers to a cell with the ability to reconstitute through multiple differentiation steps all lineages present in the immune system such as erythrocytes, granulocytes, monocytes, mast cells, lymphocytes and megakaryocytes. HSC are self-renewing and have the capacity to maintain their pluripotency. They can be purified from bone marrow, cord blood or from peripheral blood after mobilization induced by treatment with GM-CSF. The immunophenotypic markers that define a true pluripotent hematopoietic stem cell are not completely defined and different authors focus on different subsets of markers to define the population of HSC. HSCs have been defined as CD34+, CD133+, CD34−/CD133+, CD34+/CD133+, CD34 −/CD38+, CD34+/CD38−, CD45+ and c-kit+. HSC preferably have the immunophenotype of c-kit CD117 positive or of c-kit CD117 and CD45 positive.

As used herein, “Stem Cell Factor” (SCF), also known as “Steel factor”, “mast cell growth factor” or “c-kit ligand” in the art, is a transmembrane protein with a cytoplasmic domain and an extracellular domain. Soluble SCF refers to a fragment cleaved from the extracellular domain at a specific proteolytic cleavage site. SCF is well known in the art; see European Patent Publication No. 0423980A1, corresponding to European Application No. 90310889.1.

As used herein, c-kit CD117 refers to the stem cell factor receptor transmembrane molecule from mammalian species. C-kit is also known as CD117, PBT, SCFR, KIT, kit oncogene, v-kit Hardy Zuckerman 4 feline sarcome viral oncogene homolog. In mice, the c-Kit proto-oncogene is the cellular homolog of the transforming gene of a feline retrovirus (v-Kit). The c-kit protein includes characteristics of a protein kinase transmembrane receptor. In humans, KIT encodes the human homolog of the proto-oncogene c-kit. C-kit was first identified as the cellular homolog of the feline sarcoma viral oncogene v-kit. KIT is a type 3 transmembrane receptor for SCF.

As used herein, the term “growth factors” is art recognized and is intended to include all factors that are capable of stimulating the growth of a cell, maintaining the survival of a cell and/or stimulating the differentiation of a cell. Therefore the term growth factor includes without limitation one or more of platelet derived growth factors (PDGF), e.g., PDGF AA, PDGF BB; insulin-like growth factors (IGF), e.g., IGF-I, IGF-II; fibroblast growth factors (FGF), e.g., acidic FGF, basic FGF, .beta.-endothelial cell growth factor, FGF 4, FGF 5, FGF 6, FGF 7, FGF 8, and FGF 9; transforming growth factors (TGF), e.g., TGF-β1, TGF-.beta.1.2, TGF-.beta.2, TGF-.beta.3, TGF-.beta.5; bone morphogenic proteins (BMP), e.g., BMP 1, BMP 2, BMP 3, BMP 4; vascular endothelial growth factors (VEGF), e.g., VEGF, placenta growth factor; epidermal growth factors (EGF), e.g., EGF, amphiregulin, betacellulin, heparin binding EGF; interleukins, e.g., IL-1, IL-2, IL-3, IL-4, IL-5, IL-6, IL-7, IL-8, IL-9, IL-10, IL-11, IL-12, IL-13, IL-14; colony stimulating factors (CSF), e.g., CSF-G, CSF-GM, CSF-M; nerve growth factor (NGF); stem cell factor; hepatocyte growth factor, and ciliary neurotrophic factor. The term encompasses presently unknown growth factors that may be discovered in the future, since their characterization as a growth factor will be readily determinable by persons skilled in the art. Also suitable are alternative ligands and antibodies that bind to the respective cell-surface receptors for the aforementioned factors.

“Leukemia Inhibitory Factor” (LIF), is also known as DIA or differentiation inhibiting activity. LIF and uses of LIF are also well known in the art; see for example Gearing et al, U.S. Pat. No. 5,187,077 and Williams et al, U.S. Pat. No. 5,166,065. It should be recognized that SCF and LIF are all proteins and as such certain modifications can be made to the proteins which are silent and do not remove the activity of the proteins as described herein. Such modifications include additions, substitutions and deletions. Also, these proteins can be purified from animal tissues of different species or synthetically produced by DNA recombinant technology and have an amino acid sequence corresponding to SCF or LIF proteins native to different animal species such as human, baboon, mouse, etc.

As used herein, “major histocompatibility complex” or “MHC” refers to the major histocompatibility complex of class I and class II molecules involved in the presentation of antigens to T cells. Class I MHC molecules are expressed in nearly all nucleated cells and consist of a heavy chain linked to a small invariant protein called β2-microglobulin. There are three class I genetic loci in humans (A, B and C) and two in mice (K and D). Class II MHC molecules, which consist of a α and β glycoprotein chain are expressed only by antigen presenting cells. There are three class II genetic loci in humans (DR, DP, DQ) and two in mice (IA, IE). Each class II locus encompasses an alpha and beta gene, which respectively encode the α and β chains. Both class I and class II MHC genes are highly polymorphic, and are co-dominantly expressed in each cell. Consequently, each nucleated cell expresses multiple class I MHC molecules, and multiple class II MHC molecules can be expressed on antigen presenting cells.

As used herein, “genetically mismatched” or “allogeneic” refers to a genetic mismatch between class I and/or class II MHC molecules expressed between the recipient of the transplanted cells and the donor cells.

As used herein, “immunotolerance” refers to an inhibition of a graft recipient's immune response which would otherwise occur, e.g., in response to the introduction of a nonself MHC or HLA antigen into the recipient subject. Immunotolerance can involve humoral, cellular, or both humoral and cellular responses. Immunotolerance, as used herein, refers not only to complete immunologic tolerance to an antigen, but to partial immunologic tolerance, i.e., a degree of tolerance to an antigen which is greater than what would be seen if a method of the invention were not employed. Immunotolerance also refers to a donor antigen-specific inhibition of the immune system as opposed to the broad spectrum inhibition of the immune system seen with immunosuppressants. Immunotolerance is the ability of the graft to survive in an allogeneic recipient subject without chronic immunosuppression.

As used herein, the term “undifferentiated” when applied to ESC refers to morphological characteristics of undifferentiated cells, clearly distinguishing them from differentiated cells of embryo or adult origin. Undifferentiated ESC are easily recognized by those skilled in the art, and typically appear in the two dimensions of a microscopic view in colonies of cells with high nuclear/cytoplasmic ratios and prominent nucleoli. It is understood that colonies of undifferentiated cells within the population will often be surrounded by neighboring cells that are differentiated.

As used herein, the term “feeder cells” or “feeders” are used to describe cells of one type that are co-cultured with cells of another type, to provide an environment in which the cells of the second type can grow. For example, certain types of embryonic stem cells can be supported by primary mouse embryonic fibroblasts, immortalized mouse embryonic fibroblasts, or human fibroblast-like cells differentiated from human embryonic stem cells. Cell populations are said to be “essentially free” of feeder cells if the cells have been grown through at least one round after splitting in which fresh feeder cells are not added to support the growth of the adult hematopoietic stem cells.

As used herein, the phrase “therapeutically effective amount” refers to the amount of adult hematopoietic stem cells in a selected population sufficient to show a meaningful patient benefit, i.e., treatment, healing, prevention or amelioration of the relevant medical condition, or an increase in rate of treatment, healing, prevention or amelioration of such conditions.

As used herein, the term “teratoma” refers to undifferentiated embryonic stem cells that are administered to a recipient subject that lead to a jumble of cell types which form a type of tumor (Pedersen, R. A.: Embryonic stem cells for medicine. Sci. Amer. 280: 68-73, 1999). One of skill in the art would recognize the occurrence of such a tumor.

The present invention provides methods for producing ESCs that are induced to differentiate into adult stem cells, particularly into adult hematopoietic stem cells (HSCs), which are then selected for those cells displaying c-kit CD117 and for their use in functional reconstitution of the immune system in partially or totally myeloablated subjects.

In one embodiment, the present invention provides an isolated population of adult hematopoietic stem cells. These cells are differentiated from ESC and cells that are c-kit CD117 positive are selected using techniques known to those in the art. This population is capable of proliferating in culture. In another embodiment, the isolated population of adult hematopoietic stem cells that are c-kit CD117 positive and capable of proliferating in culture are produced by culturing an embryonic stem cell in a medium that comprises at least one growth factor so that a population of cells is formed. From that population, cells displaying a c-kit CD117 cell surface specific marker are selected. In one aspect, the selected cells are least 1% c-kit CD117 positive.

The present inventors contemplate that one of skill in the art would know how to produce or obtain embryonic stem cells. For example, embryonic stem cells can be prepisolated from blastocysts of members of the primate species (Thomson et al., Proc. Natl. Acad. Sci. USA 92:7844, 1995) and human embryonic stem (hES) cells can be prepared from human blastocyst cells using the techniques described by Thomson et al. (U.S. Pat. No. 5,843,780; Science 282:1145, 1998; Curr. Top. Dev. Biol. 38:133 ff., 1998) and Reubinoff et al, Nature Biotech. 18:399, 2000.

In another embodiment, the present invention contemplates that ESC are expanded without promoting ESC differentiation. Accordingly, if desired, embryonic stem cells may be expanded prior, concurrently or subsequent to differentiating the ESC into adult hematopoietic stem cells. Techniques for culturing and promoting stem cell growth without promoting differentiation are known in the art. ESCs can be propagated continuously in culture, using culture conditions that promote proliferation without differentiation by several techniques. These include but are not limited to, for example, culturing ESCs in a medium that contains inhibition factor (LIF). Alternately, ESC populations may be expanded without differentiation by culturing ESC on a layer of feeder cells, typically fibroblasts derived from embryonic or fetal tissue. The fibroblasts may be irradiated or treated with mitomycin C and cultured in the presence of lymphocyte inhibition factor (LIF). Stromal support cells for feeder layers may include embryonic bone marrow fibroblasts, bone marrow stromal cells, fetal liver cells, or cultured embryonic fibroblasts (see U.S. Pat. No. 5,690,926). Additionally, ESC can be maintained in an undifferentiated state even without feeder cells. The environment for feeder-free cultures includes a suitable culture substrate, particularly an extracellular matrix such as Matrigel.RTM. or laminin. The ESCs are plated at >15,000 cells cm.sup.-2 (optimally 90,000 cm.sup.-2 to 170,000 cm.sup.-2). Typically, enzymatic digestion is halted before cells become completely dispersed (say, about 5 min with collagenase IV). Clumps of about 10-2000 cells are then plated directly onto the substrate without further dispersal. Feeder-free cultures are supported by a nutrient medium typically conditioned by culturing irradiated primary mouse embryonic fibroblasts, telomerized mouse fibroblasts, or fibroblast-like cells derived from ESC. Examples are illustrated in the Carpenter, U.S. Pat. No. 6,833,269, herein incorporated by reference.

In accordance with the present invention, a population of cells are obtained by culturing, differentiating ESC in the presence of growth factors that enrich the cells with the desired phenotype of displaying a c-kit CD117 cell surface marker.

In one embodiment, ESC can be differentiated in vitro or ex vivo by culturing the ESC in the presence of at least one growth factor. Suitable growth factors include without limitation one or more of platelet derived growth factors (PDGF), e.g., PDGF AA, PDGF BB; insulin-like growth factors (IGF), e.g., IGF-I, IGF-II; fibroblast growth factors (FGF), e.g., acidic FGF, basic FGF, beta-endothelial cell growth factor, FGF 4, FGF 5, FGF 6, FGF 7, FGF 8, and FGF 9; transforming growth factors (TGF), e.g., TGF-PI, TGF-.beta. 1.2, TGF-.beta.2, TGF-beta3, TGF-beta.5; bone morphogenic proteins (BMP), e.g., BMP 1, BMP 2, BMP 3, BMP 4; vascular endothelial growth factors (VEGF), e.g., VEGF, placenta growth factor; epidermal growth factors (EGF), e.g., EGF, amphiregulin, betacellulin, heparin binding EGF; interleukins, e.g., IL-1, IL-2, IL-3, IL-4, IL-5, IL-6, IL-7, IL-8, IL-9, IL-10, IL-11, IL-12, IL-13, IL-14; colony stimulating factors (CSF), e.g., CSF-G, CSF-GM, CSF-M; nerve growth factor (NGF); stem cell factor; hepatocyte growth factor, and ciliary neurotrophic factor. The term encompasses presently unknown growth factors that may be discovered in the future, since their characterization as a growth factor will be readily determinable by persons skilled in the art. Also suitable are alternative ligands and antibodies that bind to the respective cell-surface receptors for the aforementioned factors. The present inventors contemplate that the growth factors may be endogenous or exogenous to the medium and/or to the ESC.

In yet another embodiment, ESC are differentiated into a population of adult hematopoietic stem cells that are c-kit CD117 positive by culturing the ESC in vitro or ex vivo in a medium that comprises at least one growth factor selected from the group consisting of: stem cell factor (SCF), interleukin-3 (IL-3), and interleukin-6 (IL-6).

If desired, the embryonic stem cells can be differentiated in vitro or ex vivo, either by culturing with a growth factor, such as a SCF, IL-3 or IL-6, or by withdrawing one or more factors that prevent ESC differentiation, for example LIF. In a preferred embodiment of the present invention, differentiation of the ESC into HSC is induced in vitro by withdrawal of LIF and culturing the ESC onto methylcellulose in growth medium supplemented with IL-3, IL-6 and SCF.

Differentiated adult hematopoietic cells can be characterized according to a number of phenotypic criteria. The criteria include but are not limited to microscopic observation of morphological features, detection or quantitation of expressed cell markers, enzymatic activity, or their receptors, for example, CD45 and c-kit cell surface markers, and electrophysiological function. As shown in Example 2, the present inventors have demonstrated that c-kit is not found on undifferentiated cells. Assays for embryonic stem cell differentiation (which will identify, among others, proteins that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, M. G. In Culture of Hematopoietic Cells. R. I. Freshney, et al. eds. Vol pp. 265-268, Wiley-Liss, Inc., New York, N.Y. 1994; Hirayama et al., Proc. Natl. Acad. Sci. USA 89:5907-5911, 1992; Primitive hematopoietic colony forming cells with high proliferative potential, McNiece, I. K. and Briddell, R. A. In Culture of Hematopoietic Cells. R. I. Freshney, et al. eds. Vol pp. 23-39, Wiley-Liss, Inc., New York, N.Y. 1994; Neben et al., Experimental Hematology 22:353-359, 1994; Cobblestone area forming cell assay, Ploemacher, R. E. In Culture of Hematopoietic Cells. R. I. Freshney, et al. eds. Vol pp. 1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Freshney, et al. eds. Vol pp. 163-179, Wiley-Liss, Inc., New York, N.Y. 1994; Long term culture initiating cell assay, Sutherland, H. J. In Culture of Hematopoietic Cells. R. I. Freshney, et al. eds. Vol pp. 139-162, Wiley-Liss, Inc., New York, N.Y. 1994.

The unique isolated cells of the present invention are separated from other cells by virtue of their c-kit CD117 cell surface markers. Advantageously, selection for c-kit CD117 alone is easier to perform than the double selection of markers. Furthermore, the recovery of cells is higher as it only involves one step of selection instead of two consecutive steps. (Examples 6, 7 and 12). The cells can be isolated by conventional techniques for separating cells, such as those described in Civin, U.S. Pat. Nos. 4,714,680, 4,965,204, 5,035,994, and 5,130,144, Tsukamoto et al U.S. Pat. No. 5,750,397, and Loken et al, U.S. Pat. No. 5,137,809, all of which are hereby incorporated by reference in their entirety. Thus, for example, a c-kit CD117-specific monoclonal antibody can be immobilized, such as on a column or on magnetic beads. The entire cell population may then be passed through the column or added to the magnetic beads. Those which remain attached to the column or are attached to the magnetic beads, which may then be separated magnetically, are those cells which contain a marker which is recognized by the antibody used. Thus, if the anti-c-kit CD117 antibody is used, then the resulting population will be greatly enriched in c-kit CD117 cells. C-kit CD117 antibodies are commercially available from several sources, for example, Research Diagnostics, Inc (Flanders, N J), eBioscience (San Diego, Calif.).

In another embodiment, the present invention provides a cell population positive for CD117 and CD45 cell surface markers. The population having c-kit CD117 cell surface markers may then be enriched in another marker by repeating the steps using a solid phase having attached thereto an antibody to the other marker CD45. Antibodies to CD45 are also commercially available. In yet another embodiment, the selected cells are at least 1% positive for c-kit CD117 and at least 1% positive for CD45.

Another technique to sort c-kit CD117 cells is by means of flow cytometry, most preferably by means of a fluorescence-activated cell sorter (FACS), such as those manufactured by Becton-Dickinson under the names FACScan or FACSCalibur. By means of this technique, the cells having a c-kit CD117 marker thereon are tagged with a particular fluorescent dye by means of an anti-c-kit CD117 antibody which has been conjugated to such a dye. This method may also be employed to isolate a population of cells of HSC that are c-kit CD117 and CD45 positive.

In addition to tagging cells having a c-kit CD117 marker thereon with a particular fluorescent dye, the CD45 cell surface marker of the cells may be tagged with a different fluorescent dye by means of an anti-CD45 antibody which is conjugated to another dye. When the stained cells are placed on the instrument, a stream of cells is directed through an argon laser beam that excites the fluorochrome to emit light. This emitted light is detected by a photo-multiplier tube (PMT) specific for the emission wavelength of the fluorochome by virtue of a set of optical filters. The signal detected by the PMT is amplified in its own channel and displayed by a computer in a variety of different forms-e.g., a histogram, dot display, or contour display. Thus, fluorescent cells which emit at one wavelength, express a molecule that is reactive with the specific fluorochrome-labeled reagent, whereas non-fluorescent cells or fluorescent cells which emit at a different wavelength do not express this molecule but may express the molecule which is reactive with the fluorochrome-labeled reagent which fluoresces at the other wavelength. The flow cytometer is also semi-quantitative in that it displays the amount of fluorescence (fluorescence intensity) expressed by the cell. This correlates, in a relative sense, to the number of the molecules expressed by the cell.

Upon induction of differentiation under these conditions, cells are stained with antibodies recognizing the c-kit CD117 and optionally CD45 surface markers and separated by fluorescence activated cell sorting (FACS). After sorting by FACS, the c-kit CD117 and optionally c-kit CD117/CD45⁺ subpopulation of cells are administered to a pre-conditioned recipient subject, where they recapitulate a multilineage hematopoietic differentiation program that results in the reconstitution of a competent immune system. Any other method for isolating a c-kit CD117 population of adult HSC as a starting material, such as bone marrow, peripheral blood or cord blood, may also be used in accordance with the present invention. The various subpopulations of the present invention may be isolated in similar manners.

The method of the current invention can find applications in several areas of modern medicine and research. An application of the present method in mammals would remove the need to find genetically matched bone-marrow donors for recipients with leukemia, immune deficiencies, autoimmune diseases and recipients that need marrow reconstitution after intense cancer chemotherapy or irradiation.

The isolated cell population of this invention can be used in therapeutic methods, such as stem cell transplantation, as well as other therapeutic methods described below, as well as others that are readily apparent to those skilled in the art. The present invention discloses a method for reconstituting or supplementing hematopoietic cell function in a recipient subject using the population of differentiated ESC selected for displaying c-kit CD117 described supra. In another aspect, the population of cells are obtained by selecting for cells that display both c-kit CD117 and CD45.

In one embodiment, a therapeutically effective amount of the selected cell population is administered into a mammalian recipient subject in need of reconstitution or supplementation. The present inventors have demonstrated that ESCs induced to differentiate ex vivo into HSCs and sorted for c-kit CD117+ or c-kit CD117+ and CD45+ reconstitute long-term multilineage hematopoiesis with a functional immune system. Examples 4, 5, 6, 7 and 12. In a preferred embodiment, the population is injected into a bone marrow cavity in a therapeutically effective amount to reconstitute the recipient's hematopoietic and immune system. The inventors contemplate that sites of injection include without limitation an intra osseous space of long bones, for example, a tibia or an iliac crest of a recipient subject. In another embodiment, the selected population is administered by intravenous route to a recipient subject requiring a bone marrow transplant to reconstitute the recipient subject's hematopoietic and immune system. Precise, effective quantities can be readily determined by those skilled in the art and will depend, of course, upon the exact condition being treated by the therapy. In many applications, however, an amount containing approximately the same number of stem cells found in one-half to one liter of aspirated marrow should be adequate.

In another embodiment, the selected cells that are at least 1% c-kit CD117 positive are used to reconstitute a recipient subject. In another embodiment, the selected cells that are at least 1% c-kit CD117 positive and at least 1% CD45 positive are used to reconstitute a recipient subject. Determination of an effective amount is well within the capability of those skilled in the art, especially in light of the detailed disclosure provided herein. A therapeutically effective amount can be estimated initially from appropriate in vitro assays and in vivo models. The therapeutically effective amount can readily be determined by routine optimization procedures.

In yet another embodiment, the population of cells is administered into a pre-conditioned recipient subject. Such preconditioning may include but is not limited to gamma or X-irradiation, immunosuppressive agents, cyclophosphamide, methylprednisolone or other chemotherapeutic drugs.

In another embodiment, the population of selected cells is injected into a myeloablated recipient subject. The present inventors contemplate that the recipient subject may be partially or totally myeloablated.

In one embodiment, the selected population of adult HSCs displaying c-kit CD117 or c-kit CD117/CD45 cell surface markers are mammalian in origin. In yet another embodiment, the selected population of adult HSCs displaying c-kit CD117 or c-kit CD117/CD45 cell surface markers are human in origin. Alternately, in a preferred embodiment, the selected population of adult HSCs are murine in origin.

The present invention also provides a method for promoting immunotolerance in a mammalian recipient subject. Adult hematopoietic stem cells produced by culturing an embryonic stem cell in a medium comprising at least one of the following: stem cell factor, interleukin-3 or interleukin-6. From these differentiated cells, cells that are c-kit CD117 positive are then selected using methods described supra. The selected c-kit CD117 positive cells are administered to a mammalian recipient subject. The present inventors contemplate that the selected population of adult HSCs displaying a c-kit CD117 cell surface marker can be syngeneic or allogeneic to the recipient subject. In another aspect, the population of cells displaying both c-kit CD117 and CD45 cell surface markers are selected and used in conjunction with the present method. In one aspect, the cells are at least 1% c-kit CD117 positive. In another embodiment, the selected cells are at least 1% c-kit CD117 positive and at least 1% c-kit CD45 positive. An important contribution of the present invention is the discovery that the methods can be used with allogeneic donor/recipient subjects without causing GVHD/HVGD or teratomas. (Examples 4 and 5). This indicates a mutual immunotolerance of the selected population of donor cells and of the remaining, if any, immune cells in the recipient subject, which results in the establishment of a stable and functional chimeric immune system, without the need of chronic immunosuppressive treatments. The present inventors also contemplate that the co-transplantation of a population of differentiated ESC and selected HSCs displaying a c-kit CD117 cell surface marker along with other tissue of the same histocompatibility type of a donor ESCs but different from the histocompatibility type of a myeloablated recipient subject will promote acceptance of the second tissue in the absence of immunosuppression, for example, hepatocytes plus hematopoietic stem cells for treating liver disease; heart muscle plus hematopoietic stem cells for treating heart disease. By creating hematopoietic chimeras improved acceptance of tissues with similarly matched MHC type can be obtained.

The present invention also discloses a method of preventing or decreasing cell mediated graft versus host disease (GVHD) derived from an allogeneic donor in a mammalian recipient subject of the transplant. GVHD is disease associated with significant morbidity caused by a pathological reaction to a bone marrow transplant in which the lymphocytes of the donated bone marrow destroy the “foreign” cells of the recipient subject. One of skill in the art would recognize the occurrence of GVHD. Manifestations of GVHD include a skin rash, an abnormality in liver function studies, fever, general symptoms including fatigue, anemia, etc.

Thus, a population of ESC differentiated into adult hematopoietic stem cells which are positive for c-kit CD117 and optionally for CD45 cell surface markers, can restore the production of hematopoietic cells to a recipient subject in need of such cells. As stated previously, the present method may be employed in treating a number of diseases and disorders. These include but are not limited to a recipient subject suffering from an autoimmune disease such as autoimmune diabetes type I, an immunodeficiency such as severe-combined immunodeficiency (SCID) or human immunodeficiency virus (HIV), a hematopoietic malignancy such as acute myeloid leukemia or other lymphoid malignancies, a non-malignant genetic disorder in hematopoiesis, for example, sickle cell anemia or thalassemia. Accordingly, the present invention may be used to replace a defective hematopoietic system in a mammalian recipient subject with a functional one.

In another embodiment, a population of these isolated cells is administered to a recipient subject to prevent insulitis and overt autoimmune diabetes type I. The method of the present invention has been successfully employed to prevent the development of autoimmune diabetes by intra bone marrow or intravenous injection of MHC-mismatched ESC-derived HSCs, without development of GVHD or host vs. graft disease and in the absence of immunosuppressive chemotherapy. (Example 6). Accordingly, an isolated population of cells provided by the present invention may be administered to a mammalian recipient subject in need thereof an effective amount to restore production of hematopoietic cells to treat a variety of diseases and disorders.

In another embodiment, the ESCs can be genetically modified to confer a particular phenotype of interest in the HSCs and/or in the terminally differentiated cells of the different hematopoietic lineages. For example, ESCs may be genetically modified to express genes that inhibit replication of HIV-1, such as ribozymes, antisense RNAs, RNA decoys, siRNAs, defective interfering viruses, or a combination thereof (42) and used to reconstitute an HIV-1-resistant immune system in immunosuppressed AIDS patients.

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention. All documents (e.g., publications and patent applications) cited herein are incorporated by reference to the same extent as if each individual document was specifically and individually indicated to be incorporated by reference.

### EXAMPLES

### Example 1

### Injection of Genetically Mismatched, Undifferentiated ESCs into Lethally Irradiated Mice

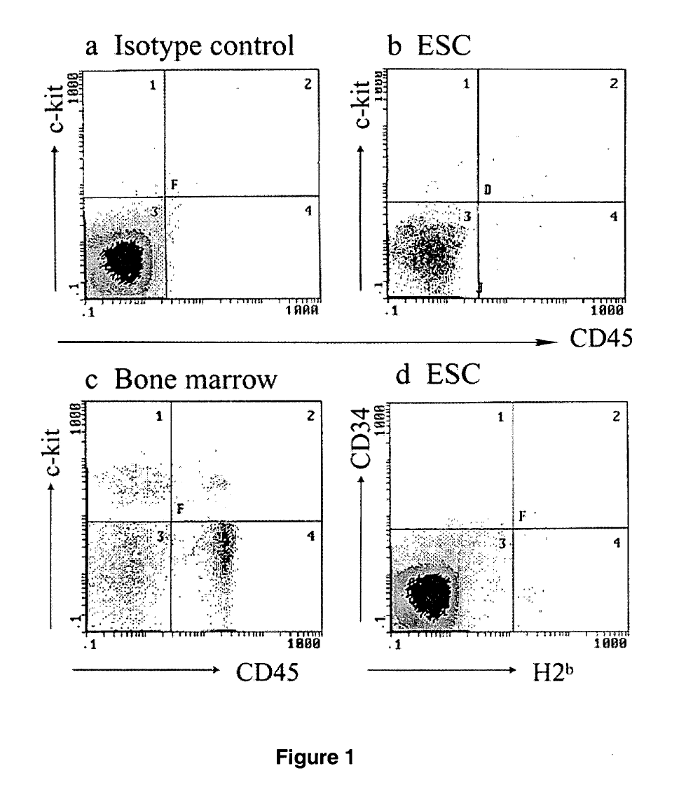

Most of the data concerning ESC-derived differentiation is based on in vitro studies (11, 12, 16, 24-27). The question of whether hematopoietic progenitors derived in vitro from mouse ESCs can support in vivo long-term multilineage engraftment remains unanswered (15, 28). Previous reports suggest that ESCs or cells derived from ESCs have a limited capacity to engraft and reconstitute hematopoiesis in vivo (15). Some components of hematopoiesis have also been reconstituted in immune-deficient mice, e.g., SCID or RAG-1-deficient mice (11, 30, 31). However, it has not been previously demonstrated that genetically normal (i.e., nontransduced) ESCs or cells derived from ESCs are capable of reconstituting an intact and functional immune system in normal mice. To investigate these questions, murine ESCs cultured under different conditions were injected into lethally irradiated mice and tested for functional immune reconstitution. Murine ESCs were maintained in the undifferentiated state by co-culture on irradiated primary fibroblasts in the presence of LIF. Flow cytometric analysis of undifferentiated ESCs showed the absence of CD117 (c-kit), CD45, CD34, or MHC molecules on their surface (FIG. 1, a-d). When undifferentiated ESCs were injected either intravenously (i.v.) or intra bone marrow (IBM) into lethally irradiated mice, marrow/hematopoietic failure resulted in 100% mortality within 8-13 d. This indicates that undifferentiated ESCs do not have the capacity to develop hematopoietic precursors in vivo able to repopulate the marrow and regenerate a fully functional multilineage immune system, and indicates that ex vivo differentiation of ESCs with the appropriate combination of growth factors and cytokines is necessary to achieve a successful transplant.

### Example 2

### Immunophenotype of Ex Vivo Cytokine-Stimulated Hematopoietic Differentiation of ESCs

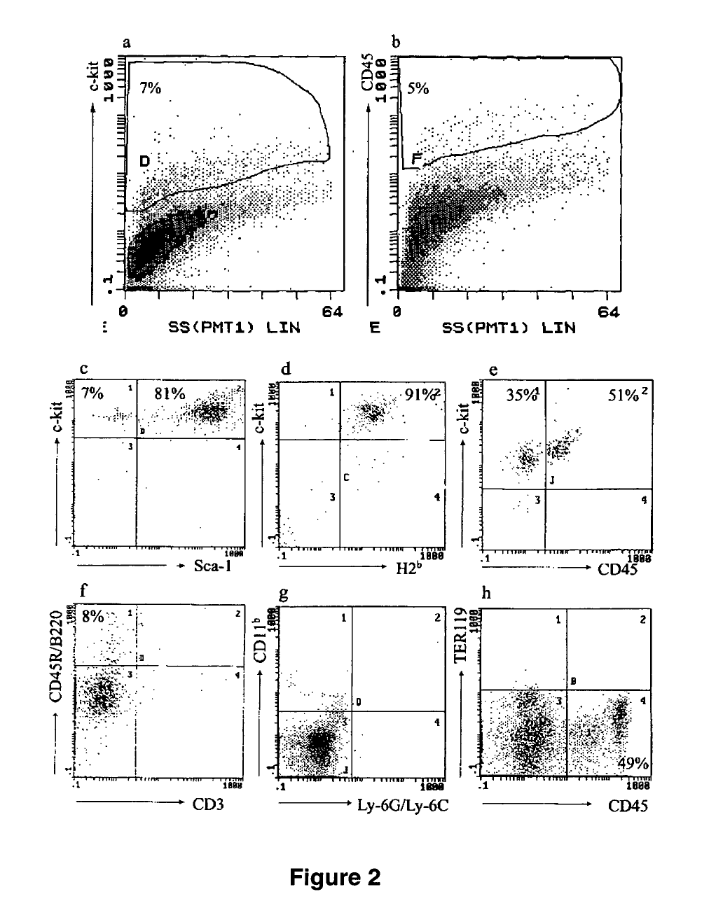

To promote ex vivo hematopoietic differentiation, undifferentiated ESCs were cultured in methylcellulose medium by the withdrawal of LIF and the addition of the hematopoietic cytokines SCF, IL-3, and IL-6 for 7-10 d resulting in formation of EB. Previous data suggested that multipotent, long-term, repopulating hematopoietic progenitors might be formed within EB around day 4 after LIF withdrawal (29) and that SCF and CD45 receptors arise around day 8 of EB culture (15). Miyagi et al. (30) reported that ESCs express low levels of the c-kit receptor on their surface, whereas Hole et al. (15) reported that expression of c-kit is absent in ESCs until day 8 of EB culture. Flow cytometric analysis of presorted population revealed that 7% of cultured cells presented the HSC marker c-kit and ˜5% presented the panleukocytic marker CD45 (FIG. 2, a and b). The immunophenotype of c-kit⁺ CD45⁺ ESC derived progenitor cells is Sca-1⁺ (FIG. 2c), H2^{b+} (FIG. 2d), and lineage⁻ for B cell marker B 220 (FIG. 2f), monocytes/granulocytes marker CD11b (FIG. 2g), and red blood cell marker Ter119 (FIG. 2h). In agreement with Hole et al., we found no expression of c-kit on undifferentiated ESCs. C-kit expression appeared between days 6 and 8 of ESC culture in methylcellulose medium. To avoid undifferentiated cells that may generate teratomas as well as unwanted excessive differentiation of ESCs into more mature stages, we harvested cells that had been cultured for 7-10 d.

### Example 3

### In Vitro Colony-Forming Unit Formation of Cytokine-Stimulated ESCs

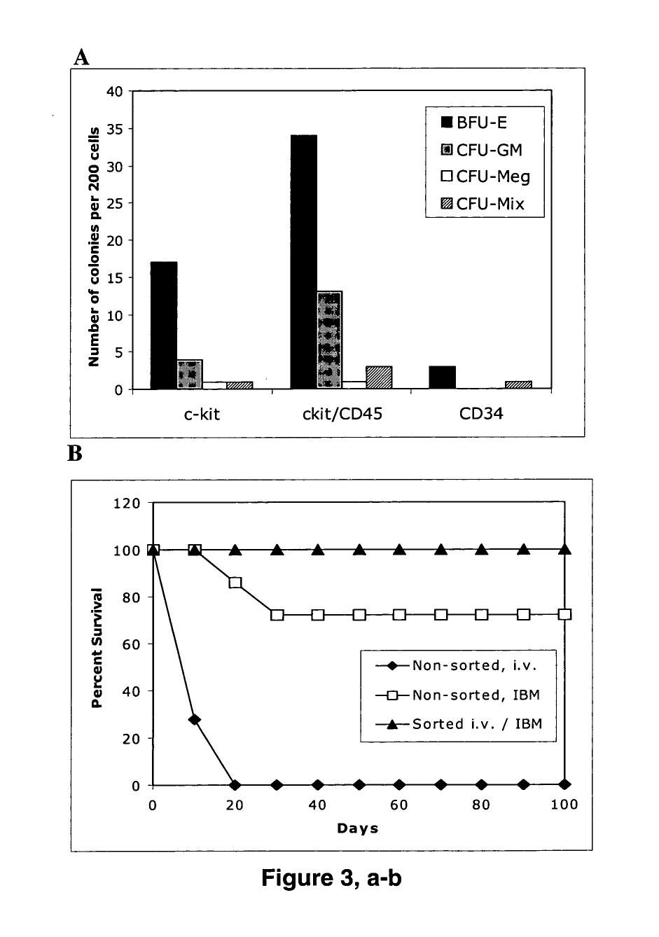

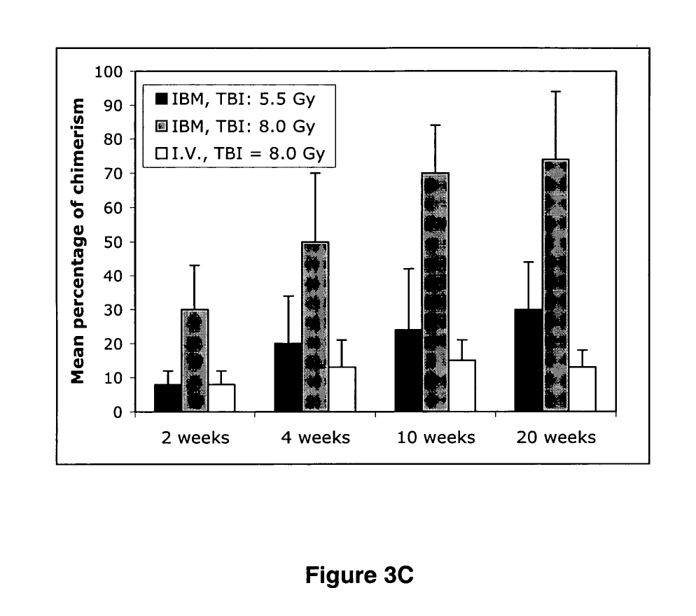

The in vitro ability of cytokine-stimulated ESCs to form hematopoietic colonies was investigated from sorted ESC-derived hematopoietic progenitor cells expressing CD34, c-kit, CD45, or both c-kit and CD45. Enriched by flow cytometry, cell subsets were plated in prepared methylcellulose-based cultures supplemented with SCF, IL-3, IL-6, and/or recombinant erythropoietin. Total progenitor frequency of colony-forming units CFU-GM, BFU-E, CFU-Meg, and CFU-Mix, was scored after 12 d of culture (FIG. 3a). The highest plating efficiency from cytokine-stimulated ESCs was observed with dual positive c-kit⁺ CD45⁺ cells that formed the largest number of CFU-GM, BFU-E, CFU-Meg, and CFU Mix colonies (FIG. 3 a). As there is no consensus regarding the immunophenotypic features of murine ESC-derived HSCs, we chose the phenotypic markers c-kit/CD45 for the subsequent purification of high efficiency repopulating cells, based on in vitro colony-forming assay that found inclusion of the CD45⁺ cell population along with c-kit⁺ cells results in more efficient in vitro functionality. Other reports have suggested that sorting for CD41 and c-kit expression may result in better enrichment of definitive hematopoietic progenitors (33, 34). Analysis of cell population sorted for CD45/c-kit cells (enriched, but not clonal) revealed enrichment for cells expressing the HSC marker Sca-1 as well as lacking lineage-specific markers. To this date, the identification and true clonal phenotype of human HSCs remains elusive. For this reason, clinical human stem cell transplants that result in long-term engraftment use a non-clonal but enriched population of marrow or blood cells selected for progenitor markers such as CD34 or CD133. As shown below, our data demonstrates that ESC-derived hematopoietic progenitor cells (enriched, but non-clonal) also result in stable, long-term hematopoietic engraftment.

### Example 4

### In Vivo Injection of Cytokine-Stimulated ESCs

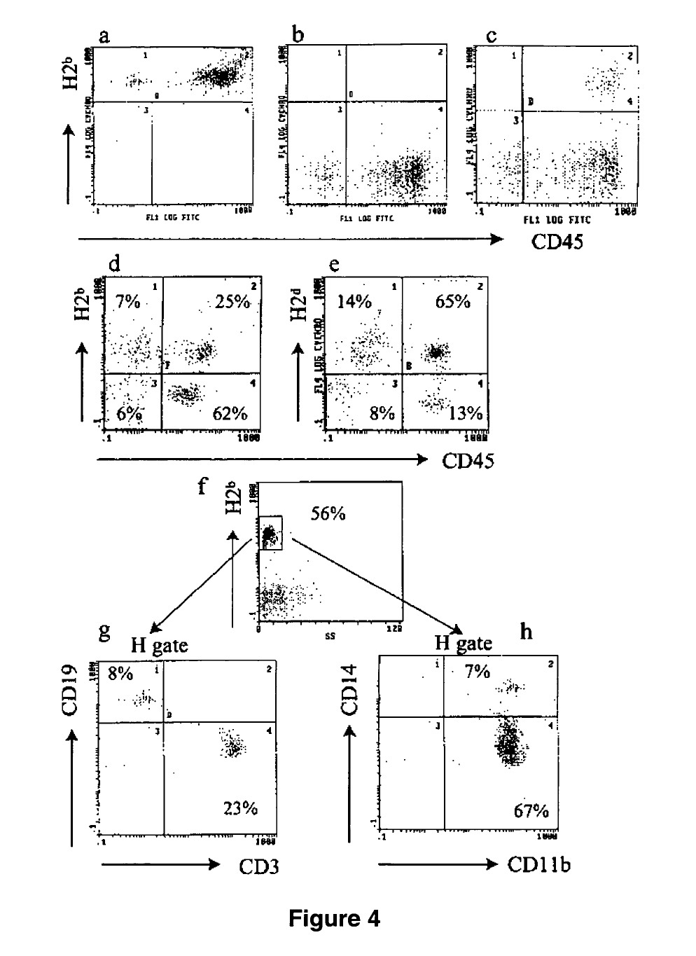

Intravenous injection of non-sorted cytokine differentiated ESCs into lethally irradiated mice did not result in hematopoietic reconstitution leading to death of all (n7) mice between days 8-13 due to bone marrow failure (FIG. 3b). In the case of IBM injection of non-sorted cytokine-differentiated ESC suspensions, hematopoiesis was reconstituted with a low percentage of donor-mixed chimerism (2-12%), however, in two out of seven mice, teratomas that were confirmed histologically arose at the IBM injection site (FIG. 3b). As already mentioned, the largest number of ex vivo hematopoietic colonies of myeloid, erythroid, and megakaryocytic lineages arose from cytokine-stimulated ESCs that were enriched for c-kit⁺ and CD45⁺ (FIG. 3 a). These two immunophenotypic markers were chosen to purify hematopoietic progenitors derived from ESCs. Therefore, ESC-derived c-kit⁺/CD45⁺ HSCs were isolated by flow cytometry and injected either i.v. (10⁶ cells in 0.2 ml) or IBM (0.5×10⁶ cells in 15 μl×2) into irradiated (TBI 5.5 or 8.0 Gy) 6-7-wk-old BALB/c mice (MHC H2^{d}; FIG. 3b). The sorted cell population prepared for injection was analyzed by flow cytometry and immunophenotypically was 86+11% c-kit+, 49±18% CD45⁺, 80-84% Sca-1⁺, ˜90% H2^{b+}, and Lin⁻ (FIG. 2, c-h). The earliest reconstitution from ESC-derived HSCs (MHC H2^{b}) was observed after 2 wk, at which time the percentage of anti-H2K^{b}/D^{b+}/CD45⁺ cells was 20.3±14.0% (Table I and FIGS. 3 c and 4, a-c). By 4 weeks after ESC-derived HSC injection, the population of H-2^{b+}/CD45⁺ cells increased to 34.4±22.4%. Analysis of chimerism performed 6 months after transplantation showed a further increase of ESC-derived hematopoiesis to 49.0+31.1% (range: 7.9-95.5%; Table I and FIG. 3c). Mice irradiated with 8.0 Gy before IBM injection of ESC-derived c-kit⁺/CD45⁺ HSCs had a higher percentage of donor chimerism compared with mice irradiated with a less immune suppressive dose (5.5 Gy) (FIG. 3 c). When comparing TBI 8.0 to 5.5 Gy, percent donor engraftment was 30.4±11.8 versus 9.2±4.2 (P=0.05) at 2 wk and 73.7±17.6 versus 30.1±13.4 (P=0.05) at 20 wk, respectively (FIG. 3 c). Mice injected IBM compared with the i.v. route of administration had faster and more effective reconstitution of hematopoiesis from ESC-derived hematopoietic progenitor cells. Between IBM and i.v. routes of administration, the percent donor engraftment was 30.4±11.8 versus 8.7±2.6 at 2 wk (P=0.05) and 73.7±17.6 versus 12.1±4.7 (P=0.01) at 20 wk, respectively (FIG. 3c). Flow cytometric analysis of PBMC subpopulations revealed that the population of donor-derived T lymphocytes (H-2^{b+}/CD3⁺ cells) comprised 18.3±4.7 and 17.3±6.5% of PBMC at 10 and 20 wk, respectively (FIG. 4, f and g). The population of H-2^{b+}/CD14⁺/CD11b⁺ (monocytes/granulocytes) was 47.3±16.5% at 10 wk after transplantation and remained stable for a maximum follow-up of 24 wk (FIG. 4, f-h). Reconstitution of chimeric B lymphocytes (H-2^{b+}/CD19⁺ cells) was 3.1±3.4% at 20 wk (FIG. 4, f and g). No mouse receiving sorted cells developed a teratoma or had evidence of malignant or abnormal growth. In summary, the data confirmed failure of hematopoietic engraftment from undifferentiated ESCs. Either i.v. or IBM injection of undifferentiated ESCs into lethally irradiated mice results in 100% mortality from marrow failure. These results also demonstrate either no or marginal hematopoietic engraftment and/or teratoma formation after injection of a non-purified heterogeneous population of cells derived from cytokine-stimulated ESCs. Intravenous injection of a cytokine-differentiated, non-sorted ESC suspension resulted in 100% mortality, whereas IBM injection resulted in only low-level chimerism (12%) and in some mice, formation of teratomas at the IBM site. However, when ESCs, induced to differentiate ex vivo into hematopoietic precursors and sorted for c-kit⁺ and CD45⁺ cells, are injected, rapid hematopoietic and immune reconstitution occurs from the ESC donor without development of teratomas. The percentage of ESC derived hematopoiesis was greater after IBM injection compared with i.v. injection despite 2 log fewer cells being injected IBM compared with i.v. These findings suggest that homing of stem cells to the marrow might be inefficient with the i.v. route of administration.

### Example 5

### Immunologic Competence of ESC-Derived Hematopoiesis

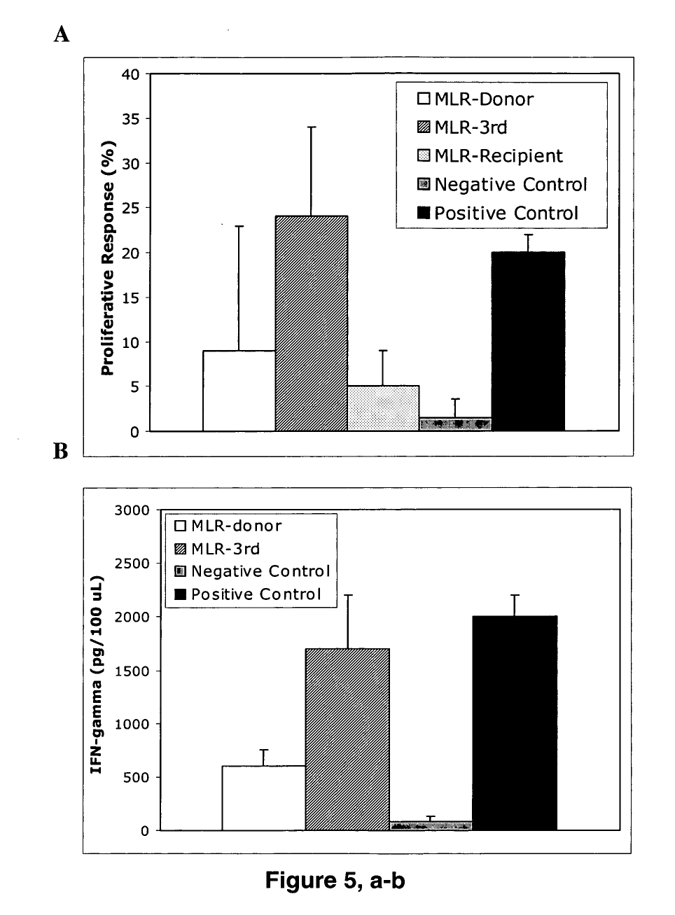

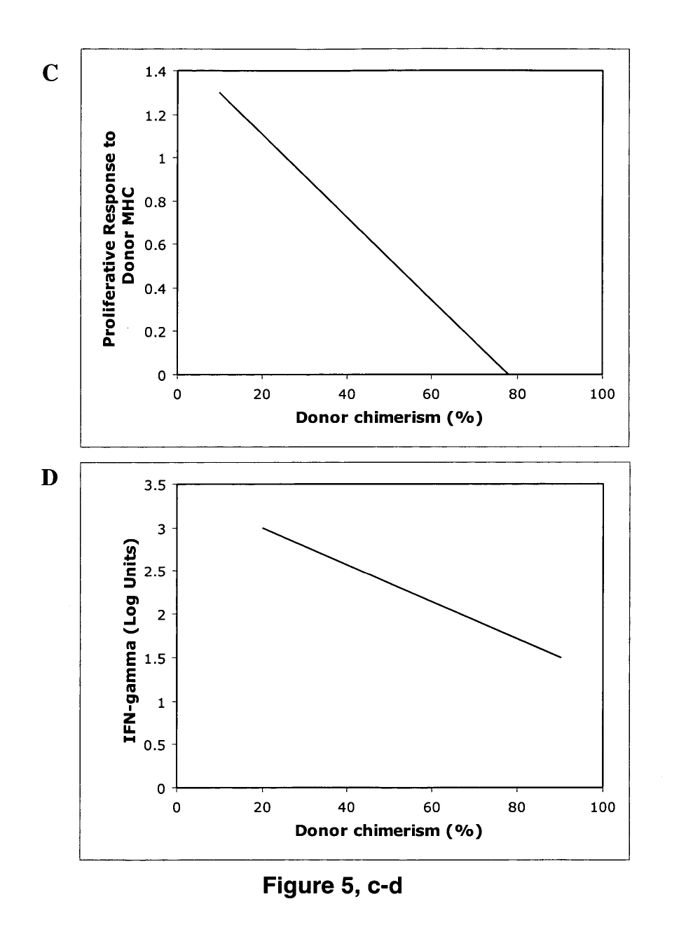

ESCs are allogeneic cells that are immunologically and genetically distinct from the recipient. However, hematopoietic reconstitution of ESC-derived T lymphocytes, B lymphocytes, and monocytes occurred across MHC barriers without evidence of rejection. No mouse developed runting (ruffled fur, hunched back, and weight loss) consistent with GVHD despite stable MHC-mismatched engraftment. There was no histological evidence of GVHD in autopsy specimens of liver or bowel. Mixed lymphocyte reactions (MLR) were evaluated by means of BrdU incorporation using splenocytes from chimeric, 129/Sv (ESC donor), BALB/c (recipient), and SJL/J (third party) mice. Splenocytes collected from chimeric mice were characterized by low MLR proliferative responses to cells of either the donor or host MHC compared with proliferative responses to SJL/J (third party, MHC-mismatched) splenocytes (9.2±2.1, 5.6±3.4, and 24.3±9.1%, respectively; FIG. 5a). IFN-γ production from chimeric mice correlated with the MLR results (FIG. 5b) in that there was an inverse correlation between percentage of donor chimerism and either proliferative response or IFN-γ production against donor genotype MHC splenocytes (R=0.89 and R=0.87, P=0.01, respectively; FIG. 5, c and d). The highest IFN-γ production against irradiated third party (SJL/J) splenocytes achieved levels of positive controls (mismatched splenocytes; 1,674.9±534.7 and 2,024.3±234.5, respectively; FIG. 5b), indicating an intact immune response to foreign antigens. In summary, in this animal model, after injection of ESC-derived hematopoietic progenitors into either the systemic circulation or IBM, we observed multilineage hematopoietic engraftment. The ESC-derived T cells were bidirectionally tolerant to recipient and host because mixed lymphocyte culture proliferative responses to recipient and host lymphocytes were diminished. This is consistent with both engraftment and absence of clinical or histological evidence of GVHD in autopsied tissues. Importantly, immune competence was maintained, as demonstrated by healthy mice without infections and normal third party MLR proliferative responses and IFN-γ production. Although some groups have previously shown transplantation of ESC-derived blood cells, engraftment was brief and/or deficient in several lineages. Our data demonstrate that ESC-derived cells enriched for a population of c-kit⁺/CD45⁺ hematopoietic progenitors may reconstitute long-term multilineage hematopoiesis with a functional immune system and without GVHD.

### Example 6

### Transplant of MHC-Mismatched ESC-Derived Hematopoietic Stem Cell Prevents Development of Diabetes in Non-obese Diabetic Mice

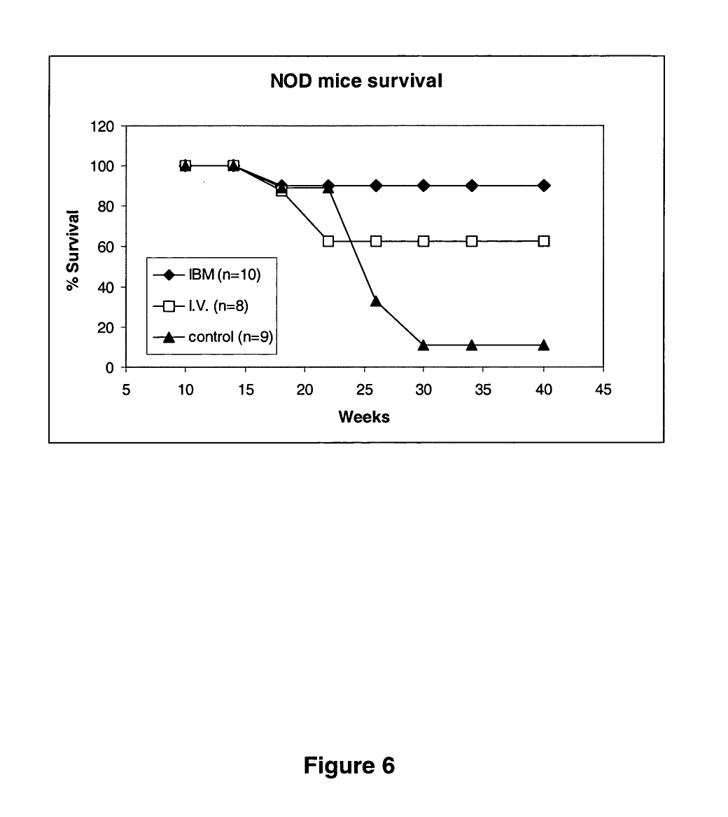

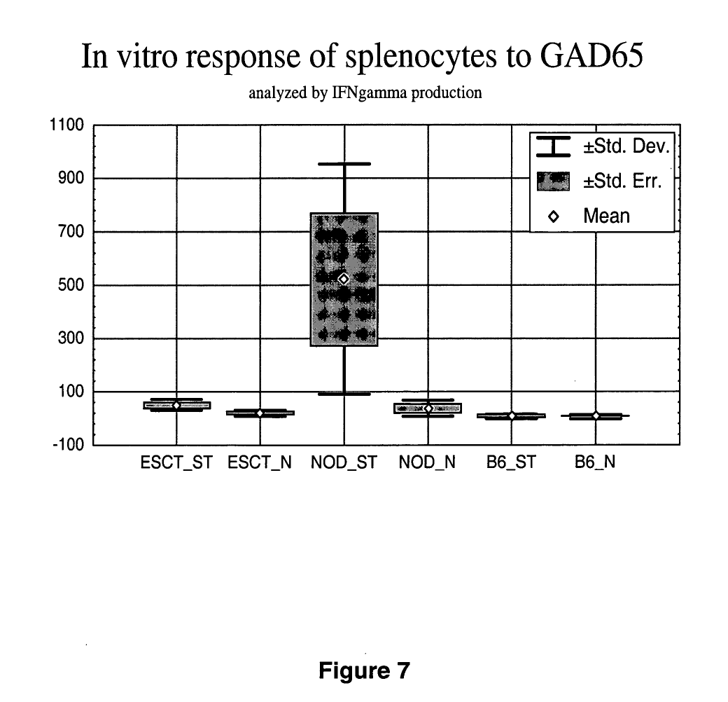

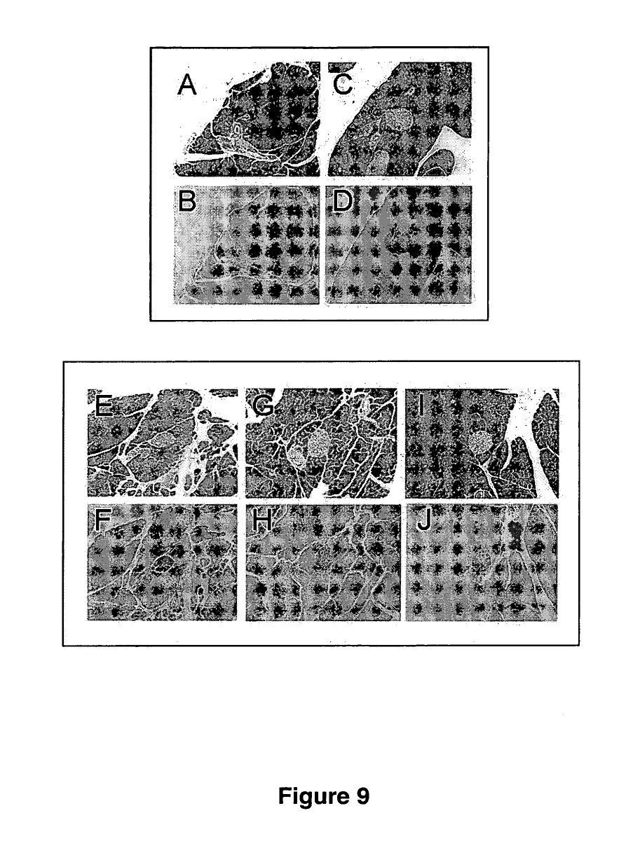

Non-obese diabetic (NOD) mice are a widely used animal model for studying type I diabetes mellitus characterized by lymphocytic infiltration of pancreatic islets followed by development of diabetes by age 3 to 4 months. It has been shown that allogeneic bone marrow transplantation can prevent insulinitis and overt diabetes. In the present invention we demonstrate that ESC-derived HSC can reconstitute bone marrow in lethally irradiated mice across MHC barriers without graft versus host disease. Another application of these cells is to induce immune tolerization by preventing autoimmune disease. We have used diabetes type I as a model of automimmune disease to demonstrate the feasibility and utility of ESC-derived HSC to prevent or treat the development of autoimmune disease. Female six-to-7 week old NOD/LtJ mice were sublethally irradiated (2×4.0 Gy) and transplanted with ESC-derived HSC. ESC were cultured in vitro as described below (Methods). Briefly, to induce differentiation toward HSC, ESC formed embryoid bodies (EB) in methylcellulose-based medium supplemented with SCF, IL-3 and IL-6. After 8-11 days, EB-derived cells were sorted for c-kit+ cells by magnetic selection using Miltenyi OctoMACS system. Suspension of HSC (92% c-kit+) was injected intra bone marrow (IBM) (5 million cells/mouse) or intravenously (10 million cells/mouse). Mice were followed by blood glucose measurements and chimerism analyses until onset of diabetes or until 40 weeks after transplantation. Nine NOD mice were held as controls. Nine out of 10 mice the from IBM group and 5 out of 8 from IV group did not become hyperglycemic in contrast to control group where 8 out of 9 mice were euthanized because of diabetes (FIG. 6). Peripheral blood donor (H2^{b}) versus recipient (H2K^{d}) chimerism was measured at 4, 10, 20, 30 weeks after transplantation using flow cytometry. The level of chimerism achieved after transplantation was 9.1%±6.71% in the IBM group and 2.5%+2.78% in the IV group. Histological examination showed that most of islets were replaced by lymphocytic infiltration or fibrous tissue in untreated controls (even in case of a mouse without clinical evidence of diabetes) (FIG. 9). In 78% (14/18) of animals from the group treated with ESC-derived HSC, remission of diabetes and lymphocitic infiltration in the pancreatic islets was confirmed by histology revealing the absence of insulitis and normal immunohistochemical staining of islet cells for insulin. Prevention of diabetes and insulitis was predicted by the percentage ESC-derived HSC chimerism. All mice with >5% ESC-derived chimerism remained free of diabetes and insulitis.

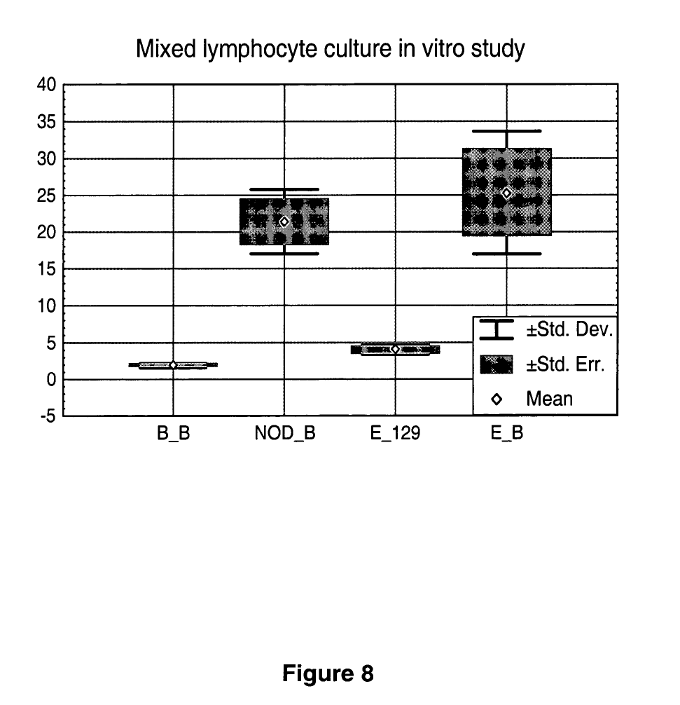

Four mice from each group were sacrificed and splenocytes were collected. Anti-GAD 65 reactivity was measured by determining interferon y level by ELISA 72h culture with or without GAD65. High concentration of IFNγ was detected only in culture containing GAD65 and splenocytes from NOD mice. IFNγ level in splenocytes cultures from NOD mice transplanted with ESC-derived HSC was comparable with negative control (FIG. 6). Immune responses in recipient NOD mice toward donor histocompatibility antigen of 129/Sv strain, recipient MHC and third party antigen were evaluated by one way mixed lymphocyte culture reaction (MLR) tests. FIG. 8 represents the data from MLRs evaluated by means of BrdU incorporation. This data indicates that proliferative response of splenocytes derived from ESC-derived HSC transplanted-NOD chimeric mice were diminished toward recipient and host lymphocytes while retaining a sustained response to third party antigens. This experiment demonstrates that the use of allogeneic ESC-derived HSC in bone marrow transplants can be used to induce immunotolerance, preventing the development of autoimmune disease, across MHC histocompatibility barriers, without the development of teratomas, graft versus host disease or host versus graft reactions, while maintaining full immunocompetence to third party antigens.

### Example 7

### Transplant of MHC-Mismatched ESC-Derived Hematopoietic Stem Cell Results in Generation of Donor-Derived Bone Marrow Stromal Cells

In the present example we demonstrate that ESC-derived HSC obtained by the method of the present invention have greater plasticity and repopulating potential than bone marrow-derived HSC and can give rise of bone marrow stromal cells. Five female six-to-7 week old BALB/c mice were sublethally irradiated (2×4.0 Gy) and transplanted with ESC-derived HSC. ESC were cultured in vitro as described below (Methods). Briefly, to induce differentiation toward HSC, ESC formed embryoid bodies in methylcellulose-based media supplemented with SCF, IL-3 and 11-6. After 8-11 days, EB-derived cells were sorted for c-kit+ cells by magnetic selection using Miltenyi OctoMACS system.

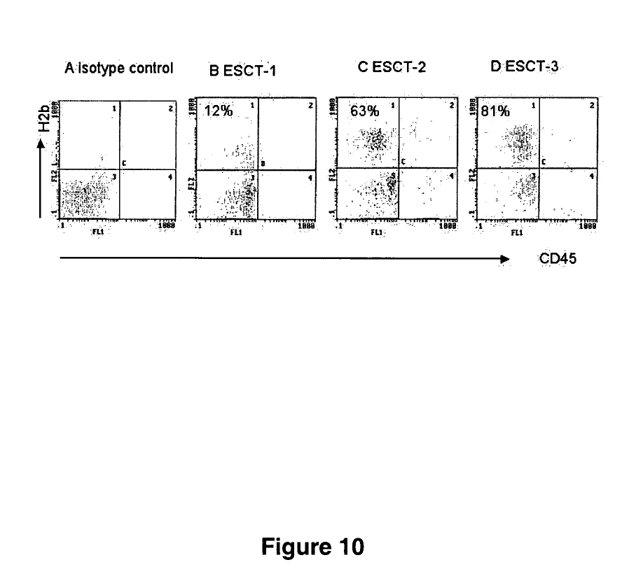

Suspension of HSC (purity: mean 90.7%, min 87%, max 95%) was injected intra bone marrow (5×10⁶ cells/mouse). Mice were followed by chimerism analyses until 10-11 weeks after transplantation when stable donor-derived chimerism was achieved. Peripheral blood and bone marrow chimerism was measured by flow cytometry: H2^{b}-donor versus H2^{d} recipient-derived. The level of peripheral blood donor chimerism was 55.4%±15.8% at 10 weeks after ESC transplantation. Mice were euthanized at 10-11 weeks after this procedure. Bone marrow cells were collected by flushing femurs and tibias with medium. In the bone marrow of transplanted animals there were 45.9%±13.1% (min 29.1%, max 61.2%) of donor-derived mononuclear cells. Bone marrow cells were cultured in high glucose DMEM supplemented with 10% FBS, 100 U/ml penicillin, 100 ug/ml streptomycin and dexamethasone 10⁻⁸ M at 37 C in 5% CO₂ atmosphere. After series of passages, attached marrow stromal cells became homogenous and devoid of hematopoietic cells. The identity of marrow stem cell (MSC) was confirmed by immunophenotypic criteria based on the absence of CD45. The proportion of CD45+ cells in marrow stem cell (MSC) population used for experiments did not exceed 2%. Expression of MHC class I antigens was very weak and difficult to analyze after standard culture conditions. In order to increase expression of MHC, MSC were pretreated with IFN gamma 100 U/ml for 72 hours prior to flow cytometry. Flow cytometric analyses of bone marrow stromal cells showed that 43.7%±33.5% (min 4.5%, max 80.9%) of cells expressed MHC of embryonic stem cells origin (FIG. 10). There was no correlation between percentage of ESC-derived chimerism in bone marrow mononuclear cells and percentage of ESC-derived mesenchymal cells expanded in vitro.

This experiment demonstrates that hematopoietic stem cells derived from ESC by the procedure described in the present invention have greater plasticity than peripheral blood or bone marrow-derived hematopoietic progenitors which are incapable of generating mesenchymal bone marrow stromal cells after transplantation. Therefore, ESC-derived HSC obtained by this method can be used as a source of bone marrow stromal cells.

### Example 8

### Preparation of ESCs

The 129/SvJX129/SV-CP F1 (MHC H2^{b}) hybrid, 3.5-d mouse blastocyst-derived ESC line R1 was provided by A. Nagy (Mount Sinai Hospital, Toronto, Canada). To maintain ESCs in an undifferentiated state they were cultured on gelatinized tissue culture dishes in high glucose Dulbecco's modified Eagle's medium supplemented with 15% FBS, 2 mM L-glutamine, 0.1 mM beta-mercaptoethanol, 1 non-essential amino acids, 1 sodium pyruvate, and 1,000 U/ml LIF (Specialty Media and StemCell Technologies Inc.). Mitomycin C-treated primary embryonic fibroblasts (StemCell Technologies Inc.) were used as a feeder layer for a long-term culture of R1 ESCs.

### Example 9

### Induction of ESCs Differentiation Toward Hematopoietic Progenitors (HSCs)

To induce differentiation toward HSCs in vitro, the ESCs were cultured on low adherent Petri dishes in Iscove's modified Dulbecco's medium containing ˜1% methylcellulose, 15% FBS, 150 μM monothioglycerol, 2 mM 1-glutamine, 500 ng/ml murine SCF, 46 ng/ml human IL-3, and 500 ng/ml human IL-6 (StemCell Technologies Inc. and Sigma-Aldrich). Cells were cultured at 37 C in 5% CO₂ atmosphere incubator for 7-10 days. The single cell suspension collected, washed, and suspended in PBS 10⁷ cells/0.2 ml for i.v. injection or 0.5×10⁷ cells/30 μl in the case of intra bone marrow (IBM) injection.

### Example 10

### Flow Cytometric Analysis

Two or three color cell cytometric analysis was performed using standard procedures on an Epics XL (Beckman Coulter). The single cell suspension was aliquoted and stained with either isotype controls or antigen-specific antibodies. Cell surface antigens were labeled with the combinations of the following monoclonal antibodies: FITC-, PE-, or biotin-(with following CyChrome staining) conjugated H2K^{b}/D^{b}, CD117 (c-kit), CD34, Sca-1, CD45, CD19, CD11b, and CD3 (BD Biosciences). Dead cells were excluded from analysis using propidium iodide staining. Samples were run on an Epics XL flow cytometer and analyzed with CELLQuest™ software (BD Immunocytometry Systems).

### Example 11

### In Vitro Hematopoietic Progenitor Assays

The single cell suspension of ESC-derived, cytokine-stimulated cells was washed and stained with the following antibodies: CD45, c-kit, and CD34. The cells were sorted using the gated dot diagrams in an Epics-Elite ESP flow cytometer cell sorter (Beckman Coulter). Four different populations of cells were used for clonal cell culture including CD34⁺ cells (purity 75%), c-kit⁺ cells (purity 63%), CD45⁺ cells (purity 75%), and a heterogeneous population consisting of CD45⁺ c-kit⁻ (12%), CD45⁻ c-kit⁺ (23%), and CD45⁺ c-kit⁺ (49%) subsets. Cells were plated in prepared methylcellulose-based cultures supplemented with a cocktail of growth factors in 35-mm Lux suspension culture dishes (Nunc) as previously described (17-19). In brief, 200 cells per 1 ml were cultured in medium containing 1.2% methylcellulose, 30% FCS (Hyclone), 1% deionized fraction V bovine serum albumin (Sigma-Aldrich), and 50 μM 2-mercaptoethanol (Sigma-Aldrich). The following colony-stimulating factors were used: 20 ng/ml murine SCF, 10 ng/ml human GM-CSF, 20 ng/ml human G-CSF, 10 ng/ml murine IL-3, 30 ng/ml murine IL-6, 3 U/ml human recombinant erythropoietin, and 100 ng/ml human TPO (StemCell Technologies Inc.). After 12 d of culture in an incubator at 37 C in humidified atmosphere with 5% CO₂, all colonies were counted under an inverted microscope. The identification of erythroid burst-forming units (BFU-E), granulocyte-macrophage colonies (CFU-GM/CFU-G/CFU-M/CFUEo), megakaryocyte colony-forming units (CFU-Meg), and erythrocyte-containing, mixed colony-forming units (CFU-Mix) colonies was based on the typical morphological features.

### Example 12

### Enrichment of ESC-Derived Hematopoietic Progenitors

Flow cytometry-based and magnetic cell sorting with microbeads were used. 1) The suspension of single cells differentiated from ESCs was collected, washed, and stained with the following antibodies: CD45 and c-kit. The cells were sorted using an Epics-Elite ESP flow cytometer cell sorter (Beckman Coulter) as a heterogeneous population consisting of CD45⁺ c-kit⁻, CD45⁻ c-kit⁺, and CD45⁺ c-kit⁺ subsets. The phenotypic purity of sorted cells determined by post-sorting flow cytometry analysis was 86±11% for c-kit and 49±18% for CD45. 2) The suspension of single cells differentiated from ESCs was collected, washed, and labeled with CD117 MicroBeads (Miltenyi Biotec). Cell expressing the mouse CD117 (c-kit) antigen were positively selected using OctoMACS system according to the manufacture's protocol (Miltenyi Biotec). After sorting, cells were resuspended in PBS and used immediately for IBM (10⁵ cells/30 μl) or i.v. injection (10⁶ cells/0.2 ml).

### Example 13

### Long-Term Repopulation Model

Mice. 6-7-wk-old female BALB/cJ mice (MHC H2^{d}; Jackson ImmunoResearch Laboratories) were used as recipients of both ESCs and cytokine induced ESCs. Mice were irradiated (total body irradiation [TBI] 5.5 or 8.0 Gy) 16 h before injection. Female six-to-7 week old NOD/LtJ were purchased from Jackson Labs and used as recipients of ESC-derived HSCs after TBI 2×4.0 Gy. The mice were housed in microisolator cages under specific pathogen-free conditions and provided with γ-irradiated food in the animal facilities of Northwestern University. All animal experiments were approved by the Institutional Animal Care and Use Committee of Northwestern University.

### Example 14

### Transplantation

Cells prepared as described above were injected either i.v. or IBM. i.v. injection was performed into one of the lateral tail veins. IBM injection was performed according to a previously described procedure (20). In brief, mice were anesthetized and after shaving and disinfection, a 5-mm incision was made on the thigh. The knee was flexed to 90 degrees and the proximal side of the tibia was drawn anteriorly. A 26-gauge needle was inserted into the joint surface of the tibia through the patellar tendon and advanced into the bone marrow cavity. Using a 50-μl microsyringe (Hamilton), the cells were injected through the bone hole and into the bone marrow cavity. The skin was then closed using 6-0 vicryl sutura (Ethicon).

### Example 15

### Chimerism

The presence of donor-derived (R1 ESC, H2^{b}) T lymphoid, B lymphoid, monocytic, and granulocytic lineage was determined using flow cytometric analysis of mononuclear cells isolated from peripheral blood of mice 2, 4, 8, 12, and 20 wk after infusion of ESC-derived cells. Cell surface antigens were labeled with the following monoclonal antibodies: FITC-, PE-, or biotin-conjugated H2K^{b}/D^{b}, H2K^{b}, H2^{d}, CD45, CD45R/B220, CD19, CD11b, CD14, and CD3 (BD Biosciences). Mononuclear cells isolated from the peripheral blood of an untreated BALB/c mouse were used as a negative control. Mononuclear cells from a 129/Sv mouse served as a positive control (see FIG. 4, a-c).

### Example 16

### In Vitro Mixed Lymphocyte Reaction (MLR)

Immune responses in recipient BALB/cJ mice toward donor histocompatibility antigen of 129/Sv strain, recipient MHC, and third party antigens were evaluated by one way MLR tests. MLR tests were performed in six animals transplanted with ESC-derived cells 6 mo after transplantation. 10⁶ splenocytes from chimeric mice were cultured separately in 24-well plates (Falcon; BD Labware) with 10⁶ irradiated splenocytes (30 Gy) obtained from 129/Sv, BALB/cJ, and SJL/J (H2S) mice. Cells were cultured in a total volume of 2 ml RPMI 1640 medium (Cellgro; Mediatec) supplemented with 2 mM L-glutamine, 10 mM Hepes, 1 mM sodium pyruvate, 0.1 mM nonessential amino acids, 50 μg/ml gentamicin, and 10% FSC. After 48 and 72 h of culture in a 37 C humidified CO₂ incubator, cells were pulsed with bromodeoxyuridine (BrdU), adding 20 μg per each 2-ml well as previously described (21). 20 h after the second pulse of BrdU, cells were harvested and processed with a BrdU Flow Kit (BD Biosciences) according to the manufacturer's protocol. Cells were stained with FITC anti-BrdU and 7-amino-actinomycin. Flow cytometric data were acquired using an Epics XL flow cytometer and analyzed with CELLQuest™ software. Syngeneic and allogeneic splenocytes were used as negative and positive controls, respectively.

### Example 17

### IFN-γ Level Analysis

Spleen cells were isolated from surgically removed spleen of mice transplanted with ESC-derived cells and passed over nylon wool columns. 5×10⁵ (in 0.2 ml culture medium) chimeric splenocytes were cultured in presence of irradiated (30 Gy) donor, recipient, or mismatched (SJL/J) splenocytes in 96-well plates for 72 h. Culture supernatants were collected and levels of IFNγ in supernatants were determined by ELISA kit according to the manufacturer's protocol (R&D Systems).

### Example 18

### Collection and Expansion of Bone Marrow Stromal Cells

Bone marrow cells were collected by flushing femurs and tibias with medium. Cells were cultured in high glucose DMEM supplemented with 10% FBS, 100 U/ml penicillin, 100 ug/ml streptomycin and dexamethasone 10⁻⁸ M at 37 C in 5% CO2 atmosphere. After a series of passages, attached marrow stromal cells became homogenous and devoid of hematopoietic cells. The identity of marrow stromal cells (MSC) was confirmed by immunophenotypic criteria based on the absence of CD45. The proportion of CD45+ cells in MSC population used for experiments did not exceed 2%.

MHC class I antigens were not present after standard culture conditions. In order to increase expression of MHC, MSC were pretreated with IFN gamma (Peprotech, Rochy Hills, NJ) 100 U/ml for 72 hours prior to flow cytometry.

### Example 19

### Grading of Histological Changes of GVHD

All mice were killed 6 months after ESC-derived transplantation (ESCT). For evaluation of presence and degree of hepatic and intestinal inflammation, tissues were removed from all mice in both groups and kept in 10% formaldehyde. Tissue sections were embedded in paraffin, sectioned, and stained with hematoxylin and eosin by standard procedures. The degree of inflammation of liver and small bowel was graded in a 0-4 scale as previously described (22).

### Example 20

### Statistical Analysis

All data are presented as the mean ± standard error of the mean. Two groups of data ere analyzed by the Mann-Whitney U test (Student's t test for nonparametric distribution). P 0.05 was considered statistically significant.

### Example 21

### Preparation of Embryonic Stem Cells

Briefly, human blastocysts are obtained from human in vivo preimplantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (about 200 U/mL; Gibco) or by selection of individual colonies by micropipette. Clump sizes of about 50 to 100 cells are optimal.

### REFERENCES

1. Burt, R. K., H. J. Deeg, P. Rowlings, G. W. Santos, I. Khouri, and R. Champlin. (1996). Diseases. In On Call in-Bone Marrow Transplantation. R. K. Burt, H. J. Deeg, and G. W. Santos, editors. R. G. Landes Company, Georgetown, TX. 154-197.
2. Yeager, A. M. (2002). Allogeneic hematopoietic cell transplantation in inborn metabolic diseases. Ann. Hematol. 81:S16-S19.
3. Burt, R. K., A. E. Traynor, R. Craig, and A. M. Marmont. (2003). The promise of hematopoietic tem cell transplantation for autoimmune diseases. Bone Marrow Transplant. 31:521-524.
4. Wingard, J. R., G. B. Vogelsang, and H. J. Deeg. (2002). Stem cell transplantation: supportive care and long-term complications. Hematology. 2002:422-444.
5. Chao, N. J. 1996. Graft-versus-host disease. In On Call in-Bone Marrow Transplantation. R. K. Burt, H. J. Deeg, and G. W. Santos, editors. R. G. Landes Company, Georgetown, TX. 478-497.
6. Wobus, A. M., H. Holzhausen, P. Jakel, and J. Schoneich. (1984). Characterization of a pluripotent stem cell line derived from a mouse embryo. Exp. Cell Res. 152:212-219.
7. Carpenter, M. K., E. Rosler, and M. S. Rao. (2003). Characterization and differentiation of human embryonic stem cells. Cloning Stem Cells. 5:79-88.
8. Williams, R. L., D. J. Hilton, S. Pease, T. A. Willson, C. L. Stewart, D. P. Gearing, E. F. Wagner, D. Metcalf, N. A. Nicola, and N. M. Gough. (1988). Myeloid leukaemia inhibitory factor maintains the developmental potential of embryonic stem cells. Nature. 336:684-687.
9. Pease, S., P. Braghetta, D. Gearing, D. Grail, and L. Williams. (1990). Isolation of embryonic stem (ES) cells in media supplemented with recombinant leukemia inhibitory factor (LIF). Dev. Biol. 141:344-352.
10. Wiles, M. V., and G. Keller. (1991). Multiple hematopoietic lineages develop from embryonal stem (ES) cells in culture. Development. 111:259-267.
11. Gutierrez-Ramos, J. C., and R. Palacios. (1992). In vitro differentiation of embryonic stem cells into lymphocyte precursors able to generate T and B lymphocytes in vivo. Proc. Natl. Acad. Sci. USA. 89:9171-9175.
12. Keller, G., M. Kennedy, T. Papayannopoulou, and M. V. Wiles. (1993). Hematopoietic commitment during embryonic stem cell differentiation in culture. Mol. Cell. Biol. 13:473486.
13. Nakano, T., H. Kodama, and T. Honjo. (1994). Generation of lymphohematopoietic cells from embryonic stem cells in culture. Science. 265:1098-1101.
14. de Pooter, R. F., S. K. Cho, J. R. Carlyle, and J. C. Zúñiga-Pflücker. (2003). In vitro generation of T lymphocytes from embryonic stem cell-derived prehematopoietic progenitors. Blood. 102:1649-1653.
15. Hole, N., G. J. Graham, U. Menzel, and J. D. Ansell. (1996). A limited temporal window for the derivation of multilineage repopulating hematopoietic progenitors during embryonal stem cell differentiation in vitro. Blood. 88:1266-1276.
16. Daley, G. Q. (2003). From embryos to embryoid bodies. Generating blood from embryonic stem cells. Ann. N.Y. Acad. Sci. 996:122-131.
17. Kimura, T., H. Sakabe, S. Tanimukai, T. Abe, Y. Urata, K. Yasukawa, A. Okano, T. Taga, H. Sugiyama, T. Kishimoto, et al. (1997). Simultaneous activation of signals through gp130, c-kit, and interleukin-3 receptor promotes a trilineage blood cell production in the absence of terminally acting lineage specific factors. Blood. 90:4767-4778.
18. Minamigushi, H., N. Yahata, T. Kimura, H. Fujiki, S. Harada, J. Wang, K. Okuda, H. Kaneko, K. Hodohara, T. Banba, et al. 2000. Interleukin 6 receptor expression by human cord blood- or peripheral blood-derived primitive haematopoietic progenitors implies acquisition of different functional properties. Br. J. Haematol. 110:327-338.
19. Sonoda, Y., H. Sakabe, Y. Ohmisono, S. Tanimukai, S. Yokota, S. Nakagawa, S. C. Clark, and T. Abe. (1994). Synergistic actions of stem cell factor and other burst-promoting activities on proliferation of CD34⁺ highly purified blood progenitors expressing HLA-DR or different levels of c-kit protein. Blood. 84:4099-4106.
20. Kushida, T., M. Inaba, H. Hisha, N. Ichioka, T. Esumi, R. Ogawa, H. Iida, and S. Ikehara. (2001). Intra-bone marrow injection of allogeneic bone marrow cells: a powerful new strategy for treatment of intractable autoimmune diseases in MRL/lpr mice. Blood. 97:3292-3299.
21. Bontadini, A., R. Conte, A. Dinota, D. Belletti, C. Tassi, P. L. Tazzari, and M. Gobbi. (1990). Mixed lymphocyte reactions evaluated by means of bromodeoxyuridine incorporation. Haematologica. 75:7-11.
22. Nagler, A., M. Ohana, R. Alper, V. Doviner, Y. Sherman, E. Rabbani, D. Engelhardt, and Y. Ilan. (2003). Induction of tolerance in bone marrow transplantation recipients suppresses graft-versus-host disease in a semiallogeneic mouse model. Bone Marrow Transplant. 32:363-369.
23. McKinney-Freeman, S. L., K. A. Jackson, F. D. Camargo, G. Ferrari, F. Mavilio, and M. A. Goodell. (2002). Muscle-derived hematopoietic stem cells are hematopoietic in origin. Proc. Natl. Acad. Sci. USA. 99:1341-1346.
24. Cho, S. K., T. D. Webber, J. R. Carlyle, T. Nakano, S. M. Lewis, and J. C. Zúñiga-Pflücker. (1999). Functional characterization of B lymphocytes generated in vitro from embryonic stem cells. Proc. Natl. Acad. Sci. USA. 96:9797-9802.
25. Palacios, R., E. Golinski, and J. Samaridis. (1995). In vitro generation of hematopoietic stem cells from an embryonic stem cell line. Proc. Natl. Acad. Sci. USA. 92:7530-7534.
26. Nakayama, N., I. Fang, and G. Elliott. (1998). Natural killer and B-lymphoid potential in CD34⁺ cells derived from embryonic stem cells differentiated in the presence of vascular endothelial growth factor. Blood. 91:2283-2295.
27. Bigas, A., D. I. K. Martin, and I. D. Bernstein. (1995). Generation of hematopoietic colony-forming cells from embryonic stem cells: synergy between a soluble factor from NIH-3T3 cells and hematopoietic growth factors. Blood. 85:3127-3133.
28. Muller, A. M., and E. A. Dzierzak. (1993). ES cells have only a limited lymphopoietic potential after adoptive transfer into mouse recipients. Development. 118:1343-1351.
29. Perlingeiro, R. C. R., M. Kyba, and G. Q. Daley. (2001). Clonal analysis of differentiating embryonic stem cells reveals a hematopoietic progenitor with primitive erythroid and adult lymphoid-myeloid potential. Development. 128:4597-4604.
30. Miyagi, T., M. Takeno, H. Nagafuchi, M. Takahashi, and N. Suzuki. (2002). Flk1⁺ cells derived from mouse embryonic stem cells reconstitute hematopoiesis in vivo in SCID mice. Exp. Hematol. 30:1444-1453.
31. Potocnik, A. J., H. Kohler, and K. Eichmann. (1997). Hematolymphoid in vivo reconstitution potential of subpopulations derived from in vitro differentiated embryonic stem cells. Proc. Natl. Acad. Sci. USA. 94:10295-10300.
32. Schmitt, R. M., E. Bruyns, and H. R. Snodgrass. (1991). Hematopoietic development of embryonic stem cells in vitro: cytokine and receptor gene expression. Genes Dev. 5:728-740.
33. Mikkola, H. K. A., Y. Fujiwara, T. M. Schlaeger, D. Traver, and S. Orkin. (2003). Expression of CD41 marks the initiation of definitive hematopoiesis in the mouse embryo. Blood. 101: 508-516.
34. Mitjavila-Garcia, M. T., M. Cailleret, I. Godin, M. M. Nogueira, K. Cohen-Solal, V. Schiavon, Y. Lecluse, F. Le Pesteur, A. H. Lagrue, and W. Vainchenker. (2002). Expression of CD41 on hematopoietic progenitors derived from embryonic hematopoietic cells. Development. 129:2003-2013.
35. Ikehara, S. (2003). New strategies for BMT, organ transplantation, and regeneration therapy. Hematology. 8:77-81.
36. Wang, J., T. Kimura, R. Asada, S. Harada, S. Yokota, Y. Kawamoto, Y. Fujimura, T. Tsuji, S. Ikehara, and Y. Sonoda. (2003). SCID-repopulating cell activity of human cord blood-derived CD34⁺ cells assured by intra-bone marrow injection. Blood. 101:2924-2931.
37. Fandrich, F., X. Lin, G. X. Chai, M. Schulze, D. Ganten, M. Bader, J. Holle, D. Huang, R. Parwaresch, N. Zavazava, et al. (2002). Preimplantation-stage stem cells induce long-term allogeneic graft acceptance without supplementary host conditioning. Nat. Med. 8:171-178.
38. Chen U, Kosco M, Staerz U. (1992). Establishment and characterization of lymphoid and myeloid mixed-cell populations from mouse late embryoid bodies, “embryonic-stem-cell fetuses”. Proc Natl Acad Sci USA. 1992 Apr. 1;89(7):2541-5.
39. Keller G, Kennedy M, Papayannopoulou T, Wiles MV. (1993). Hematopoietic commitment during embryonic stem cell differentiation in culture. Mol Cell Biol. 1993 January; 13(1):473-86.
40. Kennedy M, Firpo M, Choi K, Wall C, Robertson S, Kabrun N, Keller G. (1997). A common precursor for primitive erythropoiesis and definitive haematopoiesis. Nature. 386(6624):488-93.
41. Biesecker L G, Emerson S G. (1993). Interleukin-6 is a component of human umbilical cord serum and stimulates hematopoiesis in embryonic stem cells in vitro. Exp Hematol.; 21(6):774-8.
42. Mautino M R and Morgan R A. (2002). Gene Therapy of HIV-1 Infection Using Lentiviral Vectors Expressing Anti-HIV-1 Genes. AIDS Patient Care & Stds. 16(1): 11-26).
43. Bongso, A., Soon-Chye, N., Sathananthan, H., Lian, N. P., Rauff, M. and Ratnam, S. (1989) Improved quality of human embryos when co-cultured with human ampullary cells. Hum. Reprod., 4, 706-713
44. Gardner, D. K., Vella, P., Lane, M. et al. (1998) Culture and transfer of human blastocysts increases implantation rates and reduces the need for multiple embryo transfers. Fertil. Steril., 69, 84-88
45. Thomson J A, J Kalishman, T G Golos, M Duming, C P Harris, R A Becker and J P Hearn. (1995) Isolation of a Primate Embryonic Stem Cell Line. Proc. Natl. Acad. Sci. USA 92:7844.
46. Thomson J. A., J. Itskovitz-Eldor, S. S. Shapiro, M. A. Waknotz, J. J. Swiergiel, V. S. Marshall, and J. M. Jones. (1998). Embryonic stem cell lines derived from human blastocysts Science 282: 1145-1147.
47. Thomson J. A. and V. S. Marshall. (1998). Primate embryonic stem cells. Curr. Top. in Dev. Biol. 38: 133-160.
48. Reubinoff B E, Pera M F, Fong C Y, Trounson A, Bongso A. (2000). Embryonic stem cell lines from human blastocysts: somatic differentiation in vitro. Nature Biotechnology; 18: 399-405.
49. Solter D, Knowles B B. (1975). Immunosurgery of mouse blastocyst. Proc Natl Acad Sci USA; 72:5099-5102.
50. Johansson, B. M., and M. V. Wiles. (1995). Evidence for involvement of activin A and bone morphogenetic protein 4 in mammalian mesoderm and hematopoietic development. Mol. Cell. Biol. 15:141-15144. Keller et al., Molecular and Cellular Biology 13:473-486, 1993;
51. McClanahan T., Dalrymple S., Barkett M., Lee F. (1993). Hematopoietic growth factor receptor genes as markers of lineage commitment during in vitro development of hematopoietic cells. Blood; 81:2903-2915.
52. Freshney, M. G. (1994). In Culture of Hematopoietic Cells. R. I. Freshney, et al. eds. Vol pp. 265-268, Wiley-Liss, Inc., New York, N.Y.
53. Hirayama H, Shih J P, Awgulewitsch A et al. (1992). Clonal proliferation of murine lymphohemopoietic progenitors in culture. Proc Natl Acad Sci USA; 89:5907-5911.
54. McNiece, I. K. and Briddell, R. A. (1994). In Culture of Hematopoietic Cells. R. I. Freshney, et al. eds. Vol pp. 23-39, Wiley-Liss, Inc., New York, N.Y.
55. Neben, S., Donaldson, D., Sieff, C., Mauch, P., Bodine, D., Ferrara, J., Yetz-Aldape, J., Turner, K. (1994). Synergistic effects of interleukin-11 with other growth factors on the expansion of murine hematopoietic progenitors and maintenance of stem cells in liquid culture. Exp. Hematol. 22,353-359.
56. Ploemacher, R. E. (1994). In Culture of Hematopoietic Cells. R. I. Freshney, et al. eds. Vol pp. 1-21, Wiley-Liss, Inc., New York, N.Y.
57. Spooncer, E., Dexter, M. and Allen, T. (1994). In Culture of Hematopoietic Cells. R. I. Freshney, et al. eds. Vol pp. 163-179, Wiley-Liss, Inc., New York, N.Y.
58. Sutherland, H. J. (1994). In Culture of Hematopoietic Cells. R. I. Freshney, et al. eds. Vol pp. 139-162, Wiley-Liss, Inc., New York, N.Y.

1. An isolated population of adult hematopoietic stem cells (HSC) that proliferates in culture, said population produced by the following method:
a) culturing an embryonic stem cell (ESC) in a medium that comprises at least one growth factor so that said cell forms a population of cells; and
b) selecting from said population, cells displaying a c-kit CD117 cell surface specific marker, thereby isolating a population of cells that are c-kit CD117 positive.

2. The population of claim 1 wherein said population is selected for cells displaying a CD45 cell surface specific marker.

3. The population of claim 1 wherein said growth factors are selected from a group consisting of at least one of the following: stem cell factor (SCF), interleukin-3 (IL-3), and interleukin-6 (IL-6).

4. The population of claim 1 wherein at least 1% of the selected population of HSC is c-kit CD117 positive.

5. The population of claim 2 wherein at least 1% of the selected population of HSC is CD45 positive.

6. The population of claim 2 wherein at least 1% of the selected population of HSC is CD45 and at least 1% of the cells are c-kit CD117 positive.

7. The population of claim 1 wherein the ESC is murine in origin.

8. The population of claim 1 wherein the ESC is human in origin.

9. A method of obtaining adult hematopoietic stem cells (HSC), comprising:
a) culturing an embryonic stem cell (ESC) in a medium comprising at least one hematopoietic growth factor; so that said cell forms a population of HSC; and
b) selecting from said population of (a) cells displaying a c-kit CD117 cell surface specific marker.

10. The method of claim 9 where the ESC is cultured onto a medium that contains a cell-supporting matrix free of marrow stromal cells.

11. The method of claim 10 where the cell-supporting matrix is methylcellulose.

12. The method of claim 9 wherein said method further comprises:
selecting a population of cells additionally displaying a CD45 cell surface specific marker.

13. The method of claim 12 wherein said selected population of cells is at least 1% CD45 positive.

14. The method of claim 9 wherein said selected population of cells is at least 1% c-kit CD117 positive.

15. The method of claim 12 wherein said selected population of cells is at least 1% CD45 positive and at least 1% c-kit CD117 positive.

16. The method of claim 9 where the selection of cells displaying the surface marker c-kit CD117 is performed using fluorescence activated cell sorting (FACS), magnetic cell sorting, column chromatography, or direct immune adherence.

17. The method of claim 9 where the ESC is of human origin and said selected population of HSC is administered to a human recipient subject.

18. The method of claim 9 where the ESC is of murine origin and said selected population of HSC is administered to a murine recipient subject

19. The method of claim 9 wherein said selected population of HSC are used in bone marrow transplantation.

20. A method of obtaining adult hematopoietic stem cells (HSC) comprising:
a) culturing an embryonic stem cell (ESC) in a medium with a growth factor selected from a group consisting of:
at least one of the following: stem cell factor (SCF), interleukin-3 (IL-3), and interleukin-6 (IL-6), so that said cell forms a population of HSC; and
b) selecting from said population of (a) cells displaying a c-kit CD117 cell surface specific marker, thereby isolating a population of cells that are c-kit CD117 positive.

21. The method of claim 20 where the ESC is cultured onto a medium that contains a cell-supporting matrix free of marrow stromal cells.

22. The method of claim 21 where the cell-supporting matrix is methylcellulose.

23. The method of claim 20 wherein said method further comprises:
selecting a population of cells additionally displaying a CD45 cell surface specific marker.

24. The method of claim 23 wherein said selected population of cells is at least 1% CD45 positive.

25. The method of claim 20 wherein said selected population of cells is at least 1% c-kit CD117 positive.

26. The method of claim 23 wherein said selected population of cells is at least 1% CD45 positive and at least 1% c-kit CD117 positive.

27. The method of claim 20 where the selection of cells displaying the surface marker c-kit CD117 is performed using fluorescence activated cell sorting (FACS), magnetic cell sorting, column chromatography, or direct immune adherence.

28. The method of claim 20 where the ESC is of human origin and said selected population of HSC is administered to a human recipient subject.

29. The method of claim 20 where the ESC is of murine origin and said selected population of HSC is administered to a murine recipient subject.

30. The method of claim 20 wherein said selected population of HSC is used in bone marrow transplantation.

31. A method of reconstituting or supplementing hematopoietic cell function in a recipient subject comprising:
(a) obtaining adult hematopoietic stem cells (HSC) comprising:
(1) culturing an embryonic stem cell (ESC) in a medium comprising at least one of the following: stem cell factor (SCF), interleukin-3 (IL-3), or interleukin-6 (IL-6), so that said cell forms a population of cells; and
2) selecting from said population of (1) cells that are c-kit CD117 positive;
(b) administering an effective amount of said selected c-kit CD117 positive cells into a mammalian recipient subject in need of reconstitution or supplementation.

32. The method of claim 31 where the ESC is cultured onto a medium that contains a cell-supporting matrix free of marrow stromal cells.

33. The method of claim 32 where the cell-supporting matrix is methylcellulose.

34. The method of claim 31 wherein said method further comprises:
selecting a population of cells additionally displaying a CD45 cell surface specific marker.

35. The method of claim 34 wherein said selected HSC are at least 1% CD45 positive.

36. The method of claim 31 wherein said selected HSC are at least 1% c-kit CD117 positive.

37. The method of claim 34 wherein said selected HSC are at least 1% CD45 positive and at least 1% c-kit CD117 positive.

38. The method of claim 31 where the selection of cells displaying the surface marker c-kit CD117 is performed using fluorescence activated cell sorting (FACS), magnetic cell sorting, column chromatography, or direct immune adherence.

39. The method of claim 31 where the ESC is of human origin.

40. The method of claim 31 where the ESC is of murine origin.

41. The method of claim 31 further comprising:
injecting the selected cells into a bone marrow of a partially myeloablated recipient subject.

42. The method of claim 31 further comprising:
injecting the selected cells into a bone marrow of totally myeloablated recipient subject.

43. The method of claim 31 wherein said selected HSC are allogeneic to said recipient subject.

44. The method of claim 31 wherein said selected HSC are syngeneic to said recipient subject.

45. The method of claim 31 wherein said recipient is in need of a bone marrow transplant.

46. The method of claim 31 where the recipient subject is a human being.

47. The method of claim 31 where the selection of cells displaying the surface marker c-kit CD117 is performed using fluorescence activated cell sorting (FACS), magnetic cell sorting, column chromatography, or direct immune adherence.

48. The method of claim 41 wherein after injection, the incidence of developing teratomas is decreased.

49. The method of claim 41 wherein after injection the incidence of developing graft versus host disease or host versus graft rejection is decreased.

50. The method of claim 31 wherein after injection immunotolerance is induced in a recipient subject.

51. The method of claim 31 where the selected HSC are administered by injecting said cells into the bone marrow cavity of the recipient subject.

52. The method of claim 51 where the selected HSC are administered by injecting said cells into a tibia or an iliac crest in the recipient subject.

53. The method of claim 31 where the selected HSC are administered intravenously to the recipient subject.

54. The method of claim 31 where the recipient subject has been previously subjected to immunosuppressive treatment.

55. The method of claim 31 in which the recipient subject is conditioned by total body irradiation.

56. The method of claim 31 in which the recipient subject is conditioned by an immunosuppressive agent.

57. The method of claim 31 in which the recipient subject suffers from autoimmunity.

58. The method of claim 57 in which the autoimmunity is type I diabetes.

59. The method of claim 31 in which the recipient subject suffers from immunodeficiency.

60. The method of claim 31 in which the recipient subject is infected with a human immunodeficiency virus.

61. The method of claim 31 in which the recipient subject has undergone chemotherapy.

62. The method of claim 31 in which the recipient subject suffers from a hematopoietic malignancy.

63. A method of promoting immunotolerance in a mammalian recipient subject to a cell population that is allogeneic to said recipient subject comprising:
(a) obtaining adult hematopoietic stem cells (HSC) produced by the method comprising:
1) culturing an embryonic stem cell (ESC) in a medium comprising at least one of the following: stem cell factor, interleukin-3 or interleukin-6, so that said cell forms a population of cells; and
2) selecting from said population of (1) cells that are c-kit CD117 positive;
(b) administering said selected c-kit CD117 positive cells into a mammalian recipient subject; thereby promoting immunotolerance to cells syngeneic to the transplanted HSC.

64. The method of claim 63 where the embryonic stem cells are cultured onto a medium that contains a cell-supporting matrix free of marrow stromal cells.

65. The method of claim 64 where the cell-supporting matrix is methylcellulose.

66. The method of claim 63 wherein said method further comprises:
selecting a population of cells additionally displaying a CD45 cell surface specific marker.

67. The method of claim 66 wherein said selected HSC are at least 1% CD45 positive.

68. The method of claim 63 wherein said selected HSC are at least 1% c-kit CD117 positive.

69. The method of claim 66 wherein said selected HSC are at least 1% CD45 positive and at least 1% c-kit CD117 positive.

70. The method of claim 63 where the selection of cells displaying the surface marker c-kit CD117 is performed using fluorescence activated cell sorting (FACS), magnetic cell sorting, column chromatography, or direct immune adherence.

71. The method of claim 63 where the ESC is of human origin.

72. The method of claim 63 where the ESC is of murine origin.

73. The method of claim 63 further comprising:
injecting the selected HSC into a bone marrow of a partially myeloablated recipient subject.

74. The method of claim 63 further comprising:
injecting the selected HSC into a bone marrow of totally myeloablated recipient subject.

75. The method of claim 63 wherein said recipient is in need of a bone marrow transplant.

76. The method of claim 63 where the recipient subject is a human being.

77. The method of claim 63 where the selected HSC are administered by injecting said cells into the bone marrow cavity of the recipient subject.

78. The method of claim 77 where the selected HSC are administered by injecting said cells into a tibia or an iliac crest in the recipient subject.

79. The method of claim 63 where the selected HSC are administered intravenously to the recipient subject.

80. A method of preventing or decreasing cell-mediated graft versus host disease (GVHD) and/or host versus graft disease (HVGD) in a recipient subject receiving allogeneic organ or tissue transplants, the method comprising:
(a) obtaining adult hematopoietic stem cells (HSC) produced by the method comprising:
1) culturing an embryonic stem cell (ESC) in a medium comprising at least one of the following: stem cell factor, interleukin-3 or interleukin-6, so that said cell forms a population of cells; and
2) selecting from said population of (1) cells that are c-kit CD117 positive;
(b) administering said selected c-kit CD117 positive cells into a mammalian recipient subject; thereby preventing or decreasing cell mediated GVHD and/or HVGD and graft rejection of the transplant.

81. The method of claim 80 where the ESC is cultured onto a medium that contains a cell-supporting matrix free of marrow stromal cells.

82. The method of claim 81 where the cell-supporting matrix is methylcellulose.

83. The method of claim 80 wherein said method further comprises:
selecting a population of cells additionally displaying a CD45 cell surface specific marker.

84. The method of claim 83 wherein said selected HSC are at least 1% CD45 positive.

85. The method of claim 80 wherein said selected HSC are at least 1% c-kit CD117 positive.

86. The method of claim 83 wherein said selected HSC are at least 1% CD45 positive and at least 1% c-kit CD117 positive.

87. The method of claim 80 where the selection of cells displaying the surface marker c-kit CD117 is performed using fluorescence activated cell sorting (FACS), magnetic cell sorting, column chromatography, or direct immune adherence.

88. The method of claim 80 where the ESC is of human origin.

89. The method of claim 80 where the ESC is of murine origin.

90. The method of claim 80 further comprising:
injecting the selected HSC into a bone marrow of a partially myeloablated recipient subject.

91. The method of claim 80 further comprising:
injecting the selected HSC into a bone marrow of totally myeloablated recipient subject.

92. The method of claim 80 wherein said recipient is in need of a bone marrow transplant.

93. The method of claim 80 where the recipient subject is a human being.

94. The method of claim 80 where the selected HSC are administered by injecting said cells into the bone marrow cavity of the recipient subject.

95. The method of claim 94 where the selected HSC are administered by injecting said cells into a tibia or an iliac crest in the recipient subject.

96. The method of claim 80 where the selected HSC are administered intravenously to the recipient subject.

97. The method of claim 80 where a donor solid organ that is MHC compatible with said selected HSC is transplanted into said recipient subject.

98. A method of treating autoimmune type I diabetes comprising:
(a) obtaining adult hematopoietic stem cells produced by the method comprising:
1) culturing an embryonic stem cell in a medium comprising at least one of the following: stem cell factor, interleukin-3 or interleukin-6, so that said cell forms a population of cells; and
2) selecting from said population of (1) cells that are c-kit CD117 positive;
(b) transplanting into a bone marrow cavity of a myeloablated mammalian recipient subject with autoimmune type I diabetes a therapeutic amount of said adult hematopoietic stem cells of (a).

99. The method of claim 98 where the ESC is cultured onto a medium that contains a cell-supporting matrix free of marrow stromal cells.

100. The method of claim 99 where the cell-supporting matrix is methylcellulose.

101. The method of claim 98 wherein said method further comprises:
selecting a population of cells additionally displaying a CD45 cell surface specific marker.

102. The method of claim 101 wherein said selected HSC are at least 1% CD45 positive.

103. The method of claim 98 wherein said selected HSC are at least 1% c-kit CD117 positive.

104. The method of claim 101 wherein said selected HSC are at least 1% CD45 positive and at least 1% c-kit CD117 positive.

105. The method of claim 98 where the selection of cells displaying the surface marker c-kit CD117 is performed using fluorescence activated cell sorting (FACS), magnetic cell sorting, column chromatography, or direct immune adherence.

106. The method of claim 98 where the ESC is of human origin.

107. The method of claim 98 where the ESC is of murine origin.

108. The method of claim 98 further comprising:
injecting the selected HSC into a bone marrow of a partially myeloablated recipient subject.

109. The method of claim 98 further comprising:
injecting the selected HSC into a bone marrow of totally myeloablated recipient subject.

110. The method of claim 98 wherein said selected HSC are allogeneic to said recipient subject.

111. The method of claim 98 wherein said recipient is in need of a bone marrow transplant.

112. The method of claim 98 where the selected HSC are administered by injecting said cells into the bone marrow cavity of the recipient subject.

113. The method of claim 112 where the selected HSC are administered by injecting said cells into a tibia or an iliac crest in the recipient subject.

114. The method of claim 98 where the selected HSC are administered intravenously to the recipient subject.
